(19) United States
(12) Patent Application Publication
(10) Pub. No.: US 2006/0026773 A1
(43) Pub. Date: Feb. 9, 2006

(54) Ortho-and/or meta-substituted N-alkylamino secondary para-phenylenediamine, composition for dyeing keratin fibers containing such a para-phenylenediamine, processes using this composition and uses thereof

(76) Inventors: Stephane Sabelle, Paris (FR); Alain Genet, Aulnay Sous Bois (FR)

Correspondence Address: Thomas L. Irving;FINNEGAN, HENDERSON, FARABOW,, GARRETT & DUNNER, L.L.P., 901 New York Avenue, N.W., Washington, DC, 20001-4413, US

(21) Appl. No.: 11/066,536

(22) Filed: Feb. 28, 2005

Related U.S. Application Data
(60) Provisional application No. 60/569,650, filed on May 11, 2004.

(30) Foreign Application Priority Data
Feb. 27, 2004 (FR) 04 02021

(51) Int. Cl. A61K 8/00

(52) U.S. Cl. 8/405

(57) ABSTRACT

Novel ortho and/or meta-substituted N-alkylamino secondary para-phenylenediamine compounds, a composition for dyeing keratin fibers, for example, human keratin fibers, such as hair, comprising, in a medium that is suitable for dyeing, at least one ortho- and/or meta-substituted N-alkylamino secondary para-phenylenediamine, a process for dyeing keratin fibers comprising applying the composition, uses of the composition, and a dyeing “kit” comprising the composition.

This application claims benefit of U.S. Provisional Application No. 60/569,650, filed May 14, 2004, the contents of which are hereby incorporated herein by reference. This application also claims benefit of priority under 35 U.S.C. § 119 to French Patent Application No. FR 0402021, filed Feb. 27, 2004, the contents of which are also hereby incorporated by reference.

Disclosed herein is a novel family of ortho-substituted and/or meta-substituted N-alkylamino secondary para-phenylenediamines and their use for dyeing keratin fibers, for example, human keratin fibers, such as hair.

It is known practice to dye keratin fibers, for example, human hair, with dye compositions comprising oxidation dye precursors, such as ortho- or para-phenylenediamines, ortho- or para-aminophenols, and heterocyclic compounds, which are generally referred to as oxidation bases. These oxidation bases are colorless or weakly colored compounds that, when combined with oxidizing products, may give rise to colored compounds by a process of oxidative condensation.

It is also known that the shades obtained with these oxidation bases may be varied by combining them with couplers or coloration modifiers. The couplers and coloration modifiers may, for example, be chosen from aromatic meta-diaminobenzenes, meta-aminophenols, meta-diphenols and certain heterocyclic compounds, such as indole compounds.

The variety of molecules used as oxidation bases and couplers makes it possible to obtain a wide range of colors.

The “permanent” coloration obtained using these oxidation dyes should moreover satisfy a certain number of requirements. For example, it should have no toxicological drawbacks and it should allow shades of the desired intensity to be obtained and have good resistance to external agents such as light, bad weather, washing, permanent waving, perspiration and rubbing.

The dyes should also allow white hairs to be covered and they should be as unselective as possible, i.e., they should allow the smallest possible differences in coloration to be produced over the entire length of the same keratin fiber, which is generally differently sensitized (i.e., damaged) between its end and its root.

The novel compositions of the present disclosure for dyeing keratin fibers, for example, human keratin fibers, such as hair, should be capable of providing at least one of the following advantages: to give strong, aesthetic and sparingly selective colorations in varied shades and to show good resistance to the various attacking factors to which the fibers may be subjected, by using at least one oxidation base chosen from ortho-substituted and meta-substituted N-alkylamino secondary para-phenylenediamines.

In addition, these compositions may have a good toxicological profile.

Disclosed herein is a family of oxidation bases chosen from ortho-substituted and meta-substituted N-alkylamino secondary para-phenylenediamine compounds, processes for synthesizing them and their uses, for example, for dyeing keratin fibers, for example, human keratin fibers, such as hair.

Further disclosed herein is a dye composition comprising at least one oxidation base chosen from ortho-substituted and meta-substituted N-alkylamino secondary para-phenylenediamine compounds, dyeing processes using the composition, uses of the composition for dyeing keratin fibers, for example, human keratin fibers, such as hair, and, for example, multi-compartment devices or dye “kits” comprising the composition.

According to one embodiment, the composition disclosed herein may make it possible, for example, to obtain very powerful, sparingly selective and fast, for example, light-fast, dyeing of keratin fibers, while at the same time avoiding the degradation of these fibers.

Other characteristics, aspects, objects and advantages of the present disclosure will emerge even more clearly on reading the description and the examples that follow.

As used herein, the term “alkyl” means a linear or branched C₁-C₁₀ radical, for example, chosen from methyl, ethyl, propyl, butyl, pentyl, hexyl, octyl, etc.

The novel compounds disclosed herein are chosen from ortho-substituted and meta-substituted N-alkylamino secondary para-phenylenediamines of formula (I): 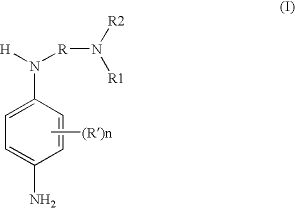
wherein:
R₁ and R₂, which may be identical or different, are each chosen from a hydrogen atom and alkyl groups, or the radicals R₁ and R₂ form, together with the nitrogen to which they are attached, a saturated 5- or 6-membered ring optionally interrupted with at least one entity chosen from nitrogen atoms and carbonyl groups, and optionally substituted with at least one alkyl group;
R is chosen from linear and branched C₂-C₁₀ alkylene radicals, optionally interrupted with at least one atom chosen from nitrogen and oxygen, and optionally substituted with at least one group chosen from amino, monoalkylamino, dialkylamino, alkylcarbonyl, amido, alkoxycarbonyl, monoalkylaminocarbonyl, and dialkylaminocarbonyl groups,
R′ is chosen from alkyl, alkoxy, alkoxyalkyl, monohydroxyalkyl and polyhydroxyalkyl groups and a chlorine atom,
n is an integer equal to 1,
with the proviso that the compound of formula (I) is not chosen from one of the following compounds N-1-(2-diethylaminoethyl)-2-methoxy-benzene-1,4-diamine; N-1-(3-diethylaminopropyl)-3-methylbenzene-1,4-diamine, and N-1-(3-pyrrolidino-propyl)-3-methyl-benzene-1,4-diamine.

In one embodiment, R₁ is chosen from a hydrogen atom and alkyl groups, R₂ being as defined above; or R₁ and R₂ form, together with the nitrogen to which they are attached, a saturated 5- or 6-membered ring optionally interrupted with at least one nitrogen atom, and optionally substituted with at least one alkyl group.

In other embodiments, R₁ is chosen from a hydrogen atom, and R₂ is chosen from a hydrogen atom and alkyl groups; or alternatively R₁ and R₂ both are chosen from alkyl groups, and, also, for example, the two alkyl groups may be identical; or alternatively R₁ and R₂ form, together with the nitrogen to which they are attached, a saturated 5- or 6-membered ring optionally interrupted with at least one entity chosen from nitrogen atoms and carbonyl groups and optionally substituted with at least one C₁-C₃ alkyl group.

In one embodiment, R is chosen from linear and branched C₂-C₈ alkylene radicals, optionally interrupted with at least one nitrogen atom.

In one embodiment, the radical R′ is chosen from C₁-C₃ alkyl, C₁-C₃ alkoxy, (C₁-C₃)alkoxy(C₁-C₃)alkyl groups and a chlorine atom.

The compounds of formula (I) may be in free form or in the form of at least one salt, such as acid addition salts, for example, chosen from hydrochlorides, hydrobromides, sulfates, citrates, succinates, tartrates, lactates, tosylates, benzenesulfonates, phosphates, and acetates.

In some embodiments, the ortho-substituted and/or meta-substituted N-alkylamino secondary para-phenylenediamine compounds of formula (I) may be chosen from compounds given in the table below:

| | |
|---|---|
| 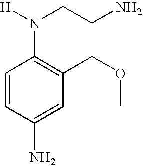 | N-1-(2-Amino-eth- yl)-2-meth- oxymethyl- benzene-1,4-di- amine |
| 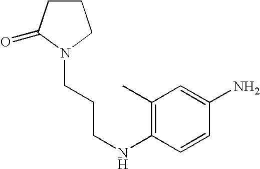 | 1-[3-(4-Amino-2-meth- ylphenyl- amino)propyl]-pyr- rolidin-2-one |
| 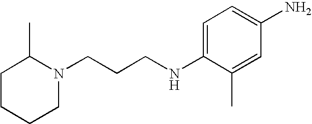 | 2-Methyl-N-1-[3-(2-meth- ylpiperid-1-yl)pro- pyl]-ben- zene-1,4-diamine |
| 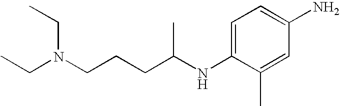 | N-1-(4-Diethyl- amino-1-methyl- butyl)-2-methyl- benzene-1,4-di- amine |
| 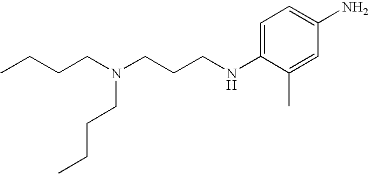 | N-1-(3-Dibutyl- aminopropyl)-2-meth- ylbenzene-1,4-di- amine |
| 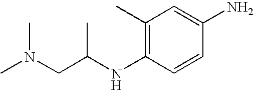 | N-1-(2-Dimethyl- amino-1-methyl- ethyl)-2-methyl- benzene-1,4-di- amine |
| 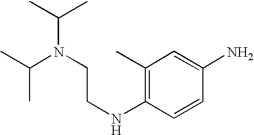 | N-1-(2-Diiso- propylamino- ethyl)-2-methyl- benzene-1,4-di- amine |
| 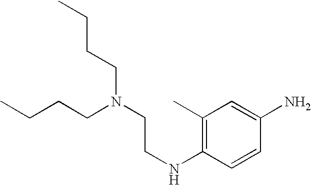 | N-1-(2-Dibutyl- aminoethyl)-2-meth- ylbenzene-1,4-di- amine |
| 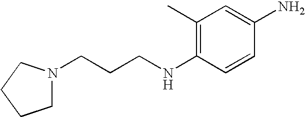 | 2-Methyl-N-1-(3-pyr- rolidin-1-yl- propyl)benzene-1,4-di- amine |
| 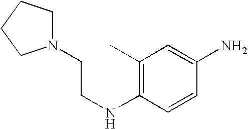 | 2-Methyl-N-1-(2-pyr- rolidin-1-yl- ethyl)benzene-1,4-di- amine |
| 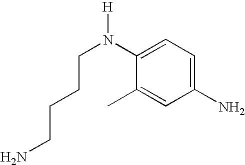 | N-1-(4-Amino- butyl)-2-methyl- benzene-1,4-di- amine |
| 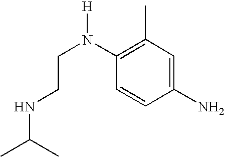 | N-1-(2-Isopro- pylaminoethyl)-2-meth- ylbenzene-1,4-di- amine |
| 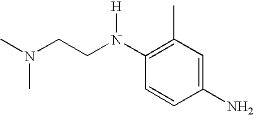 | N-1-(2-Dimethyl- aminoethyl)-2-methyl- benzene-1,4-di- amine |
| 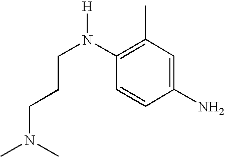 | N-1-(3-Dimethyl- aminopropyl)-2-meth- ylbenzene-1,4-di- amine |
| 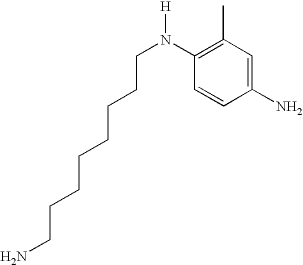 | N-1-(8-Amino- octyl)-2-methyl-ben- zene-1,4-di- amine |
| 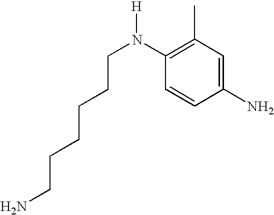 | N-1-(6-Amino- hexyl)-2-methyl- benzene-1,4-di- amine |
| 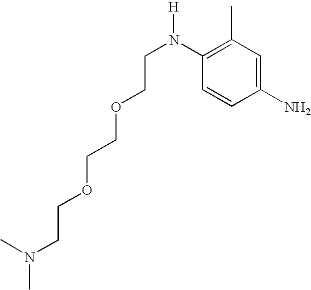 | N-1-{2-[2-(2-Di- methylaminoeth- oxy)ethoxy]-eth- yl}-2-methyl- benzene-1,4-di- amine |
| 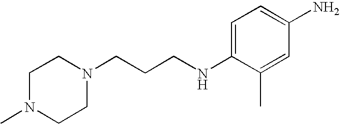 | 2-Methyl-N-1-[3-(4-meth- ylpiper- azin-1-yl)propyl]-ben- zene-1,4-di- amine |
| 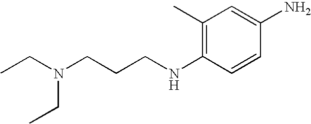 | N-1-(3-Diethyl- aminopropyl)-2-meth- ylbenzene-1,4-di- amine |
| 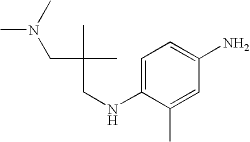 | N-1-(3-Dimethyl- amino-2,2-di- methylpropyl)-2-meth- ylbenzene-1,4-di- amine |
| 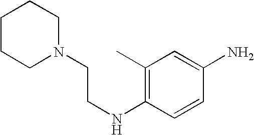 | 2-Methyl-N-1-(2-pipe- rid-1-yl- ethyl)benzene-1,4-di- amine |
| 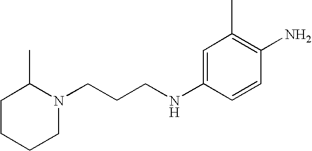 | 3-Methyl-N-1-[3-(2-meth- ylpiperid-1-yl)pro- pyl]-ben- zene-1,4-di- amine |
| 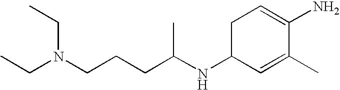 | N-1-(4-Diethyl- amino-1-methyl-bu- tyl)-3-meth- ylbenzene-1,4-di- amine |
| 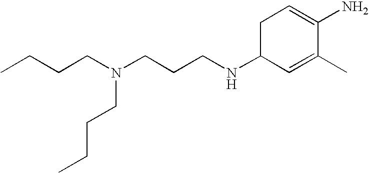 | N-1-(3-Dibutyl- aminopropyl)-2-meth- ylbenzene-1,4-di- amine |
| 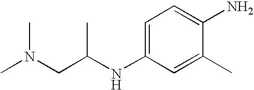 | N-1-(2-Dimethyl- amino-1-methyl- ethyl)-3-methyl- benzene-1,4-di- amine |
| 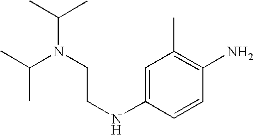 | N-1-(2-Diiso- propylaminoethyl)-3-meth- ylben- zene-1,4-di- amine |
| 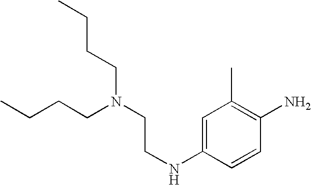 | N-1-(2-Dibutyl- aminoethyl)-3-meth- ylbenzene-1,4-di- amine |
| 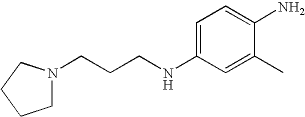 | 3-Methyl-N-1-(3-pyr- rolidin-1-yl- propyl)benzene-1,4-di- amine |
| 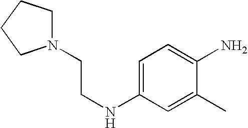 | 3-Methyl-N-1-(2-pyr- rolidin-1-yl- ethyl)benzene-1,4-di- amine |
| 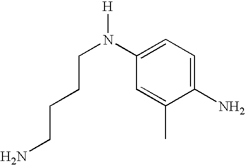 | N-1-(4-Amino- butyl)-3-methyl- benzene-1,4-di- amine |
| 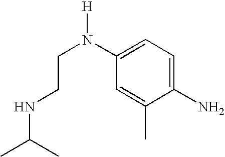 | N-1-(2-Isopro- pylaminoethyl)-3-meth- ylbenzene-1,4-di- amine |
| 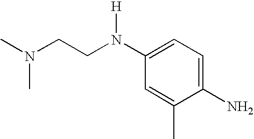 | N-1-(2-Dimethyl- aminoethyl)-3-meth- ylbenzene-1,4-di- amine |
| 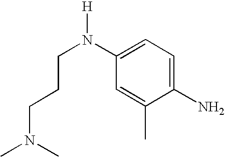 | N-1-(3-Dimethyl- aminopropyl)-3-meth- ylbenzene-1,4-di- amine |
| 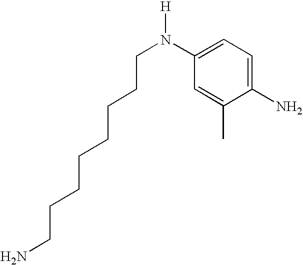 | N-1-(8-Amino- octyl)-3-methyl- benzene-1,4-di- amine |
| 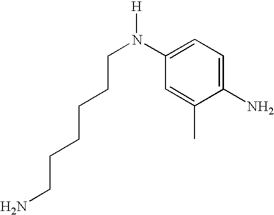 | N-1-(6-Amino- hexyl)-3-methyl- benzene-1,4-di- amine |
| 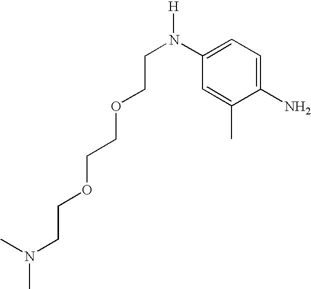 | N-1-{2-[2-(2-Di- methylaminoeth- oxy)ethoxy]-eth- yl}-3-methyl- benzene-1,4-di- amine |
| 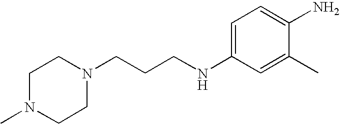 | 3-Methyl-N-1-[3-(4-meth- ylpiper- azin-1-yl)propyl]-ben- zene-1,4-di- amine |
| 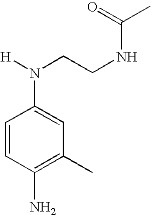 | N-1-(3-Diethyl- aminopropyl)-3-meth- ylbenzene-1,4-di- amine |
| 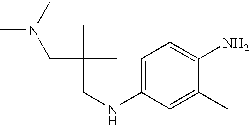 | N-1-(3-Dimethyl- amino-2,2-di- methylpropyl)-3-meth- ylbenzene-1,4-di- amine |
| 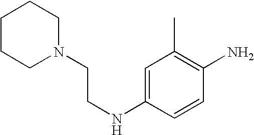 | 2-Methyl-N-4-(2-pipe- rid-1-yl- ethyl)benzene-1,4-di- amine |
| 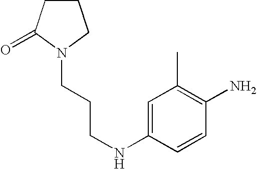 | 1-[3-(4-Amino-3-methyl- phenylamino)-pro- pyl]-pyr- rolidin-2-one |
| 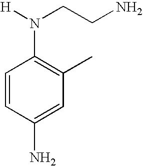 | N-1-(2-Amino- ethyl)-2-meth- ylbenzene-1,4-diamine |
| 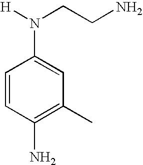 | N-4-(2-Amino- ethyl)-2-meth- ylbenzene-1,4-diamine |

The ortho-substituted and/or meta-substituted N-alkylamino secondary para-phenylenediamine compounds of formula (I) may generally be prepared according to a method comprising:
synthesis of a 4-(N-alkylamino)nitrobenzene compound by nucleophilic substitution of the halogen of a para-halonitrobenzene with a diamine of formula R₁R₂NRNH₂ (R₁, R₂ and R being as defined above) in the presence of a base,
reduction of the nitro group of the 4-(N-alkylamino)nitrobenzene compound obtained to give the compound of formula (I):

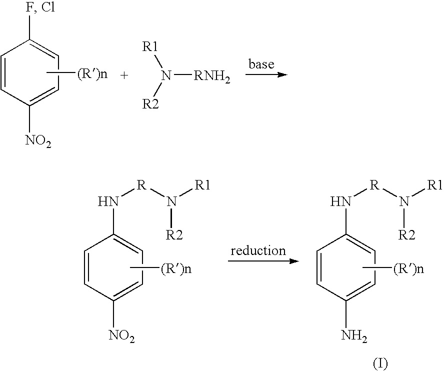

The first synthetic step is described in documents Synthesis 1990 (12), 1147-1148 and Synth. Commun. 1990, 20(22), 3537-3543.

The second step is a standard reduction step. This step is carried out by performing a hydrogenation reaction via heterogeneous catalysis in the presence of Pd/C, Pd(II)/C or Raney Nickel, or alternatively by performing a reduction reaction with a metal, for example with zinc, iron, tin, etc. (Advanced Organic Chemistry, 4^{th} edition, 1992, J. March, Wiley Interscience; Reduction in Organic Chemistry, M. Hudlicky, 1983, Ellis Honwood series Chemical Science).

A second synthetic route may be represented schematically as follows: 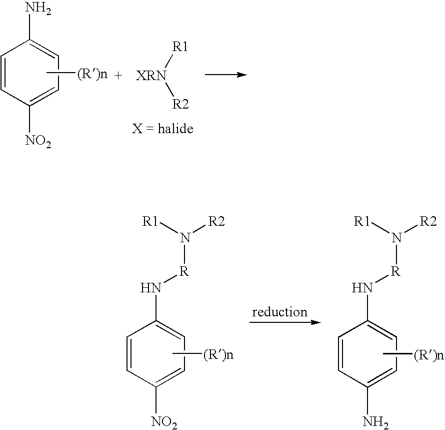
1^{st} step: coupling step inspired from J. Indian. Chem. Soc. 1990, 67, 602-603 or from Synth. Commun. 1999, 29, 1819-1933,
2^{nd} step: standard reduction step, which is performed as outlined in the general method above.

Further disclosed herein are nitro compounds of formula (II): 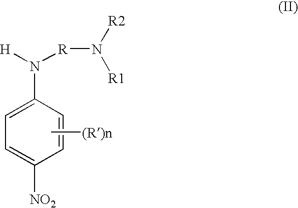
wherein:
R₁ and R₂, which may be identical or different, are each chosen from a hydrogen atom and alkyl groups, or the radicals R₁ and R₂ form, together with the nitrogen to which they are attached, a saturated 5- or 6-membered ring optionally interrupted with at least one entity chosen from nitrogen and carbonyl groups, and optionally substituted with at least one alkyl group;
R is chosen from linear and branched C₂-C₁₀ alkylene radicals, optionally interrupted with at least one atom chosen from nitrogen and oxygen, and optionally substituted with at least one group chosen from amino, monoalkylamino, dialkylamino, alkylcarbonyl, amido, alkoxycarbonyl, monoalkylaminocarbonyl, and dialkylaminocarbonyl groups;
R′ is chosen from alkyl, alkoxy, alkoxyalkyl, monohydroxyalkyl and polyhydroxyalkyl radicals, and a chlorine atom; and
n is an integer equal to 1, with the proviso that the compound of formula II is not N-1-(3-diethylaminopropyl)-3-methyl-4-nitro-1-aminobenzene and N-1(3-pyrrolidino-propyl)-3-methyl-4-nitro-1-aminobenzene and to processes for preparing the ortho-substituted and/or meta-substituted N-alkylamino secondary para-phenylenediamine compounds of formula (I) in which a reduction of the corresponding nitro compound is performed; as used here, the term “corresponding nitro compound” means the ortho-substituted and/or meta-substituted N-alkylamino secondary para-phenylenediamine compound of formula (I), wherein the amino group para to the group NHRNR₁R₂ is replaced with a nitro group.

Further disclosed herein is the use of the ortho-substituted and/or meta-substituted N-alkylamino secondary para-phenylenediamine compounds of formula (I), for dyeing keratin fibers, for example, human keratin fibers, such as hair, 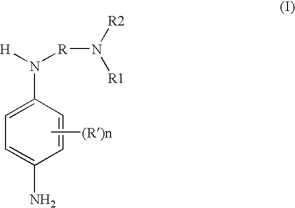
wherein:
R₁ and R₂, which may identical or different, are each chosen from a hydrogen atom and alkyl groups, or R₁ and R₂ form, together with the nitrogen to which they are attached, a saturated 5- or 6-membered ring optionally interrupted with at least one entity chosen from nitrogen atoms and carbonyl groups, and optionally substituted with at least one alkyl group;
R is chosen from linear and branched C₂-C₁₀ alkylene radicals, optionally interrupted with at least one atom chosen from nitrogen and oxygen, and optionally substituted with at least one group chosen from amino, monoalkylamino, dialkylamino, alkylcarbonyl, amido, alkoxycarbonyl, monoalkylaminocarbonyl, and dialkylaminocarbonyl groups;
R′ is chosen from alkyl, alkoxy, alkoxyalkyl, monohydroxyalkyl and polyhydroxyalkyl radicals and a chlorine atom; and
n is an integer equal to 1, with the proviso that the compound of formula II is not N-1-(3-diethylaminopropyl)-3-methyl-4-nitro-1-aminobenzene and N-1 (3-pyrroligino-propyl)-3-metyl-4-nitro-1-aminobenzene.

Further disclosed herein is a composition for dyeing keratin fibers, for example, human keratin fibers, such as hair, comprising, in a medium that is suitable for dyeing, at least one oxidation base chosen from ortho-substituted and meta-substituted N-alkylamino secondary para-phenylenediamine compounds of formula (I).

Further disclosed herein is a cosmetic composition for dyeing keratin fibers, for example, human keratin fibers, such as hair, comprising, in a medium that is suitable for dyeing, at least one oxidation base chosen from ortho-substituted and meta-substituted N-alkylamino secondary para-phenylenediamine compounds of formula (I) and at least one cosmetic adjuvant chosen from antioxidants, penetrating agents, sequestering agents, fragrances, buffers, dispersants, surfactants, conditioning agents, film-forming agents, polymers, ceramides, preserving agents, nacreous agents, opacifiers, vitamins, and provitamins.

Further disclosed herein is the use of a composition comprising, in a medium that is suitable for dyeing, at least one oxidation base chosen from ortho-substituted and meta-substituted N-alkylamino secondary para-phenylenediamine compounds of formula (I), for dyeing fibers, for example, keratin fibers, such as hair.

For example, the at least one oxidation base chosen from ortho-substituted and meta-substituted N-alkylamino secondary para-phenylenediamine compound of formula (I) may be present in an amount ranging from 0.0001% to 20% by weight, and, further, for example, from 0.005% to 6% by weight, relative to the total weight of the composition.

The medium that is suitable for dyeing may comprise water or mixtures of water and at least one organic solvent, for example, chosen from branched and unbranched C₁-C₄ lower alcohols, such as ethanol and isopropanol; polyols and polyol ethers, for example, 2-butoxyethanol, propylene glycol, propylene glycol monomethyl ether, diethylene glycol monomethyl ether and monoethyl ether; glycerol; and aromatic alcohols, such as benzyl alcohol and phenoxyethanol.

In some embodiments, the composition disclosed herein comprises at least one cosmetic adjuvant chosen from antioxidants, penetrating agents, sequestering agents, fragrances, buffers, dispersants, surfactants, conditioning agents, film-forming agents, polymers, ceramides, preserving agents, nacreous agents, opacifiers, vitamins, and provitamins.

The at least one cosmetic adjuvant may be present in an amount ranging from 0.01% to 20% by weight, relative to the weight of the total composition.

In one embodiment, the composition may further comprise at least one additional oxidation base other than the ortho-substituted and/or meta-substituted N-alkylamino secondary para-phenylenediamine compounds of formula (I). In other embodiments, the composition can further comprise at least one coupler.

The at least one coupler may be chosen, for example, from meta-phenylenediamines, meta-aminophenols, meta-diphenols, naphthalene-based couplers, heterocyclic couplers, and addition salts thereof.

Examples of the at least one coupler that may be mentioned include 2-methyl-5-aminophenol, 5-N-(β-hydroxyethyl)amino-2-methylphenol, 6-chloro-2-methyl-5-aminophenol, 3-aminophenol, 1,3-dihydroxybenzene (or resorcinol), 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene and 2,6-bis(β-hydroxyethylamino)toluene, and addition salts thereof.

The at least one coupler may be present, for example, in an amount ranging from 0.0001% to 20% by weight, and, further, for example, from 0.005% to 6% by weight, relative to the total weight of the composition.

The at least one additional oxidation base may be chosen, for example, from para-phenylenediamines, bis(phenyl)alkylenediamines, para-aminophenols, ortho-aminophenols, heterocyclic bases, and addition salts thereof.

Among the para-phenylenediamines, mention may be made, for example, of para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-amino-2-methyl-N,N-bis(β-hydroxyethyl)aniline, 4-amino-2-chloro-N,N-bis(β-hydroxyethyl)aniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-di-hydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene, 3-hydroxy-1-(4′-aminophenyl)pyrrolidine and 6-(4-aminophenylamino)hexan-1-ol, and acid addition salts thereof.

In one embodiment, among the para-phenylenediamines mentioned above, para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, 2-β-acetylaminoethyloxy-para-phenylenediamine, and acid addition salts thereof may, for example, be used.

Among the bis(phenyl)alkylenediamines, mention may be made, by way of example, of N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diaminophenoxy)-3,6-dioxaoctane, and acid addition salts thereof.

Among the para-aminophenols, mention may be made, by way of example, of para-aminophenol, 4-amino-3-methylphenol, 4-amino-3-fluorophenol, 4-amino-2-chlorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol, 4-amino-2,6-dichlorophenol, 4-amino-6[((5′-amino-2′-hydroxy-3′-methyl)phenyl)methyl]2-methylphenol, bis(5′-amino-2′-hydroxy)phenylmethane, and acid addition salts thereof.

Among the ortho-aminophenols, mention may be made, by way of example, of 2-aminophenol, 2-amino-5-methylphenol, 2-amino-6-methylphenol, 5-acetamido-2-aminophenol, and acid addition salts thereof.

Among the heterocyclic bases, mention may be made, by way of example, of pyridine derivatives, pyrimidine derivatives, and pyrazole derivatives.

Among the pyridine derivatives, mention may be made of the compounds described, for example, in Patent Nos. GB 1 026 978 and GB 1 153 196, such as 2,5-diaminopyridine, 2-(4-methoxyphenyl)amino-3-aminopyridine, and 3,4-diaminopyridine, and acid addition salts thereof.

Other pyridine oxidation bases that are useful include the 3-aminopyrazolo-[1,5-a]pyridine oxidation bases and the addition salts thereof described, for example, in Patent Application No. FR 2 801 308. Examples that may be mentioned include pyrazolo[1,5-a]pyrid-3-ylamine; 2-acetylaminopyrazolo[1,5-a]pyrid-3-ylamine; 2-morpholin-4-ylpyrazolo[1,5-a]pyrid-3-ylamine; 3-aminopyrazolo[1,5-a]pyridine-2-carboxylic acid; 2-methoxypyrazolo[1,5-a]pyrid-3-ylamine; (3-aminopyrazolo[1,5-a]pyrid-7-yl)methanol; 2-(3-aminopyrazolo[1,5-a]pyrid-5-yl)ethanol; 2-(3-aminopyrazolo[1,5-a]pyrid-7-yl)ethanol; (3-aminopyrazolo[1,5-a]pyrid-2-yl)methanol; 3,6-diaminopyrazolo[1,5-a]pyridine; 3,4-diaminopyrazolo[1,5-a]pyridine; pyrazolo[1,5-a]pyridine-3,7-diamine; 7-morpholin-4-ylpyrazolo[1,5-a]pyrid-3-ylamine; pyrazolo[1,5-a]pyrid-3,5-diamine; 5-morpholin-4-ylpyrazolo[1,5-a]pyrid-3-ylamine; 2-[(3-aminopyrazolo[1,5-a]pyrid-5-yl)-(2-hydroxyethyl)amino]ethanol; 2-[(3-aminopyrazolo[1,5-a]pyrid-7-yl)-(2-hydroxyethyl)amino]ethanol; 3-aminopyrazolo[1,5-a]pyrid-5-ol; 3-aminopyrazolo[1,5-a]pyrid-4-ol; 3-aminopyrazolo[1,5-a]pyrid-6-ol; 3-aminopyrazolo[1,5-a]pyrid-7-ol; and acid addition salts thereof.

Among the pyrimidine derivatives, mention may be made of the compounds described, for example, in Patent Nos. DE 2 359 399; JP 88 169 571; JP 05 63 124; EP 0 770 375 and Patent Application No. WO 96/15765, such as 2,4,5,6-tetraminopyrimidine, 4-hydroxy-2,5,6-triaminopyrimidine, 2-hydroxy-4,5,6-triaminopyrimidine, 2,4-dihydroxy-5,6-diaminopyrimidine and 2,5,6-triaminopyrimidine and addition salts thereof and tautomeric forms thereof, when a tautomeric equilibrium exists.

Among the pyrazole derivatives, mention may be made of the compounds described in Patent Nos. DE 3 843 892, DE 4 133 957 and Patent Application Nos. WO 94/08969, WO 94/08970, FR-A-2 733 749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and addition salts thereof.

The at least one additional oxidation base may be present in an amount ranging from 0.0001% to 20% by weight and, for example, from 0.005% to 6% by weight, relative to the total weight of the composition.

In general, the acid addition salts that can be used for the at least one additional oxidation base and the at least one coupler are chosen, for example, from hydrochlorides, hydrobromides, sulfates, citrates, succinates, tartrates, lactates, tosylates, benzenesulfonates, phosphates, and acetates.

The composition disclosed herein may also comprise at least one direct dye, which may be chosen, for example, from neutral, acidic and cationic nitrobenzene dyes; neutral, acidic and cationic azo direct dyes; neutral, acidic and cationic quinone and, for example, anthraquinone direct dyes; azine direct dyes; methine, azomethine, triarylmethane and indoamine direct dyes; and natural direct dyes. In one embodiment, the composition disclosed herein comprises at least one dye chosen from cationic direct dyes and natural direct dyes.

Among the cationic direct dyes that may be used in the composition disclosed herein, mention may be made of the cationic azo direct dyes described in Patent Application Nos. WO 95/15144, WO 95/01772 and EP 714 954.

Among these compounds, mention may be made, for example, of the following dyes:
1,3-dimethyl-2-[[4-(dimethylamino)phenyl]azo]1H-imidazolium chloride,
1,3-dimethyl-2-[(4-aminophenyl)azo]1H-imidazolium chloride, and
1-methyl-4-[(methylphenylhydrazono)methyl]pyridinium methyl sulfate.

Among the natural direct dyes, mention may be made of lawsone, juglone, alizarin, purpurin, carminic acid, kermesic acid, purpurogallin, protocatechaldehyde, indigo, isatin, curcumin, spinulosin, and apigenidin. It is also possible to use extracts or decoctions comprising these natural dyes and, for example, henna-based poultices or extracts.

The at least one direct dye may be present in an amount ranging, for example, from 0.001% to 20% by weight, and, further, for example, from 0.005% to 10% by weight, relative to the total weight of the composition.

Needless to say, a person skilled in the art will take care to select the adjuvant(s), additional oxidation base(s) and direct dye(s) such that the advantageous properties intrinsically associated with the oxidation dye composition are not, or are not substantially, adversely affected by the envisaged addition(s).

The pH of the composition may range from 3 and 12, and, for example, further from 5 to 11. It may be adjusted to the desired value using at least one agent chosen from acidifying and basifying agents usually used in the dyeing of keratin fibers and standard buffer systems.

Examples of the acidifying agents that may be used include mineral and organic acids other than carboxylic diacids, for example, hydrochloric acid, orthophosphoric acid, sulfuric acid, carboxylic acids, such as acetic acid, tartaric acid, citric acid and lactic acid, and sulfonic acids.

Examples of the basifying agents include aqueous ammonia, alkali metal carbonates, alkanolamines, such as monoethanolamine, diethanolamine and triethanolamine and derivatives thereof, sodium hydroxide, potassium hydroxide and compounds of formula: 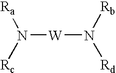
wherein W is chosen from propylene residues optionally substituted with at least one substituent chosen from a hydroxyl group and C₁-C₄ alkyl radicals; Rₐ, R_{b}, R_{c} and R_{d}, which may be identical or different, are each chosen from a hydrogen atom and C₁-C₄ alkyl and C₁-C₄ hydroxyalkyl radicals.

The composition disclosed herein may be in various forms, such as a form chosen from liquids, creams, gels, and any other forms that may be suitable for dyeing keratin fibers, for example, human hair.

Further disclosed herein is a process wherein at least one composition disclosed herein is applied, in the presence of at least one oxidizing agent, to keratin fibers for a time that is sufficient to develop the desired coloration, wherein the at least one oxidizing agent is applied before, simultaneously with or after the at least one composition. The color may be developed at acidic, neutral or alkaline pH and the at least one oxidizing agent may be added to the at least one composition at the time of use, or it may be used starting with at least one oxidizing composition comprising it, which is applied simultaneously with or sequentially to the at least one composition.

In one embodiment, the at least one composition is mixed, for example, at the time of use, with at least one oxidizing composition comprising, in a medium that is suitable for dyeing, at least one oxidizing agent, wherein the at least one oxidizing agent is present in an amount that is sufficient to develop a coloration. According to this embodiment, at least one ready-to-use composition is provided, which is a mixture of the at least one composition with the at least one oxidizing agent, for example, chosen from hydrogen peroxide, urea peroxide, alkali metal bromates, persalts, peracids and oxidase enzymes. The mixture obtained, in the form of at least one ready-to-use composition, is then applied to the keratin fibers for a time that is sufficient to develop the desired coloration. After an action time ranging from 3 to 50 minutes and, for example, 5 to 30 minutes, the keratin fibers are rinsed, washed with a shampoo, rinsed again and then dried.

The at least one oxidizing agent may be chosen from those conventionally used for the oxidation dyeing of keratin fibers. For example, the at least one oxidizing agent may be chosen from hydrogen peroxide, urea peroxide, alkali metal bromates, persalts, such as perborates and persulfates, peracids and oxidase enzymes, among which mention may be made of peroxidases, two-electron oxidoreductases such as uricases, and four-electron oxygenases, such as laccases. In one embodiment, hydrogen peroxide may be used.

The at least one oxidizing composition may also comprise at least one adjuvant conventionally used in the composition disclosed herein and as defined above.

The pH of the at least one oxidizing composition comprising the at least one oxidizing agent is such that, after mixing with the at least one dye composition, the pH of the resulting composition applied to the keratin fibers ranges, for example, from 3 to 12 and, further, for example, from 5 to 11. It may be adjusted to the desired value by means of at least one agent chosen from acidifying and basifying agents usually used in the dyeing of keratin fibers and as defined above.

The at least one ready-to-use composition that is finally applied to the keratin fibers may be in various forms, such as in the form chosen from liquids, creams, gels, and any other forms that are suitable for dyeing keratin fibers, for example, human hair.

Further disclosed herein is a multi-compartment device or dyeing “kit”, wherein at least one first compartment comprises the at least one composition defined above and at least one second compartment comprises at least one oxidizing composition. This device may be equipped with a means for applying the desired mixture to the hair, such as the devices described in Patent Application No. FR-2 586 913 in the name of the Applicant.

Using this device, it is possible to dye keratin fibers via a process that includes mixing the at least one dye composition with the at least one oxidizing agent and applying the mixture obtained to the keratin fibers for a time that is sufficient to develop the desired coloration.

Other than in the operating examples, and where otherwise indicated, all numbers expressing quantities of ingredients, reaction conditions, and so forth used in the specification and claims are to be understood as modified in all instances by the term “about.” Accordingly, unless indicated to the contrary, the numerical parameters set forth in this specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The examples that follow serve to illustrate the various embodiments disclosed herein without, however, being limiting in nature.

### EXAMPLES

### Example 1

### Synthesis of N-1-(2-aminoethyl)-2-methoxymethylbenzene-1,4-diamine trihydrochloride (25)

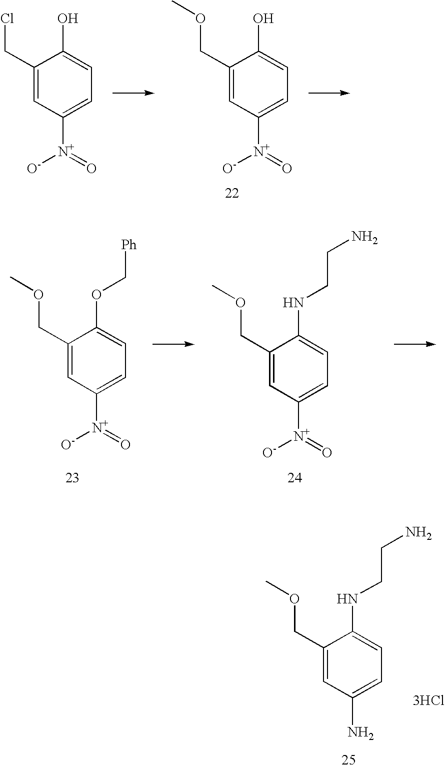

### Step 1: Preparation of 2-methoxymethyl-4-nitrophenol (22)

18.7 g (0.1 mol) of chloromethyl-2-nitro-4-phenol and 50 ml of methanol were refluxed for 2 hours 30 minutes. 37 mls (0.2 mol) of 5.4 N sodium methoxide were added dropwise to this solution. After cooling, the reaction medium was poured into 350 ml of ice-water. The light insoluble matter was filtered off and the filtrate was then acidified with 10 ml of acetic acid; the oil crystallized. 11.4 g of 2-methoxymethyl-4-nitrophenol were obtained.

### Step 2: Preparation of 1-benzyloxy-2-methoxymethyl-4-nitro-benzene (23)

12.1 ml (0.105 mol) of benzyl chloride was added over 10 minutes to a mixture of 18.3 g (0.1 mol) of 2-methoxymethyl-4-nitrophenol (22) obtained above and 8.3 g (0.06 mol) of potassium carbonate in 40 ml of dimethylformamide heated on a boiling water bath. The reaction mixture was poured onto 150 g of ice. The yellow crystals obtained were filtered off and then washed with water and recrystallized from isopropanol. 21.1 g of 1-benzyloxy-2-methoxymethyl-4-nitro-benzene was obtained (melting point=108° C.).

| | THEORY | FOUND |
|---|---|---|
| C | 65.93 | 65.86 |
| H | 5.53 | 5.56 |
| O | 23.42 | 23.62 |
| N | 5.13 | 5.19 |

### Step 3: Preparation of N-1-(2-methoxymethyl-4-nitrophenyl)ethane-1,2-diamine (24)

54.6 g (0.2 mol) of 1-benzyloxy-2-methoxymethyl-4-nitrobenzene (23) was added portionwise over 10 minutes to 150 ml of ethylenediamine, heated on a boiling water bath. After heating for 4 hours 30 minutes, the reaction medium was poured into 800 g of ice-water. The precipitate obtained was filtered off by suction and dried. 31.2 g of product was thus obtained.

| | THEORY | FOUND |
|---|---|---|
| C | 53.32 | 53.33 |
| H | 6.71 | 6.72 |
| O | 21.31 | 21.57 |
| N | 18.65 | 18.54 |

### Step 4: Preparation of N-1-(2-aminoethyl)-2-methoxymethylbenzene-1,4-diamine trihydrochloride (25)

27.15 g (0.12 mol) of N-1-(2-methoxymethyl-4-nitrophenyl)-ethane-1,2-diamine (24) was reduced in 60 g of zinc powder, 12 ml of water, 120 ml of ethanol and 2.4 g of ammonium chloride, and maintained at reflux. After filtration, the para-phenylenediamine was trapped in hydrochloride form by addition of 70 ml of 7N hydrochloric ethanol. On evaporating to dryness, 31 g of N-1-(2-aminoethyl)-2-methoxymethylbenzene-1,4-diamine was obtained.

| | THEORY | FOUND |
|---|---|---|
| C | 39.43 | 53.33 |
| H | 6.62 | 6.72 |
| O | 34.91 | 21.57 |
| N | 13.79 | 18.54 |
| Cl | 34.91 | 35.35 |

The proton NMR and mass spectra were in accordance with the expected structure of the product.

### Example 2

### Synthesis of 1-[3-(4-amino-3-methylphenylamino)propyl]-pyrrolidin-2-one dihydrochloride (27)

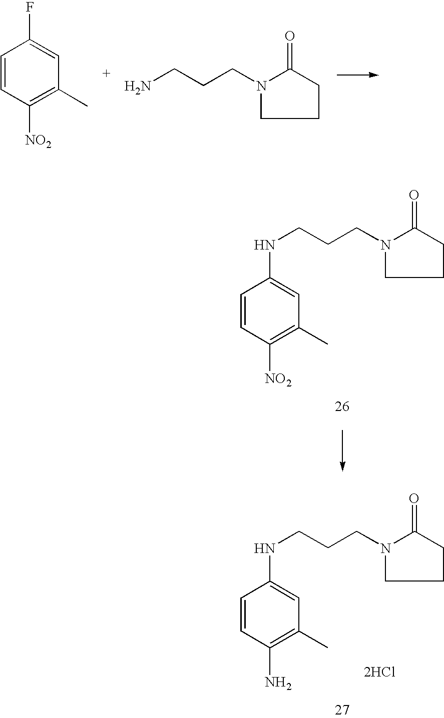

### Step 1: Synthesis of 1-[3-(4-nitro-3-methylphenylamino)propyl]pyrrolidin-2-one (26)

2 g of 5-fluoro-2-nitrotoluene was added to a solution of 20 ml of N-methylpyrrolidinone, 2.2 g of N-(3′-aminopropyl)-2-pyrrolidinone and 2.14 g of K₂CO₃. The reaction medium was heated at 60° C. for 15 hours and, after cooling to room temperature, poured into a mixture of water and ice. The yellow precipitate formed was filtered off, reslurried in water and then dried over P₂O₅. 2.8 grams of 1-[3-(4-nitro-3-methylphenylamino)propyl]pyrrolidin-2-one (26) were obtained.

### Step 2: Synthesis of 1-[3-(4-amino-3-methylphenylamino)propyl]pyrrolidin-2-one dihydrochloride (27)

The 1-[3-(4-nitro-3-methylphenylamino)propyl]pyrrolidin-2-one (26) obtained above was reduced with a boiling zinc/ammonium chloride/water/ethanol mixture. The corresponding amine was isolated in dihydrochloride form.

The proton NMR and mass spectra were in accordance with the expected structure of the product.

### Example 3

### Synthesis of N-1-(2-dimethylaminoethyl)-2-methylbenzene-1,4-diamine dihydrochloride (29)

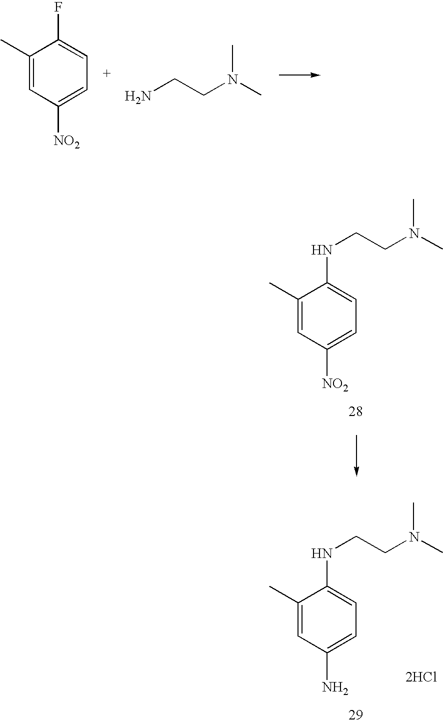

### Step 1: Synthesis of N-1-(2-dimethylaminoethyl)-2-methyl-4-nitro-1-aminobenzene (28)

2 g of 2-fluoro-5-nitrotoluene was added to a solution of 20 ml of N-methylpyrrolidinone, 1.36 g of 2-dimethylaminoethylamine, and 2.14 g of K₂CO₃. The reaction medium was heated at 60° C. for 12 hours and, after cooling to room temperature, was then poured into a water and ice mixture. The yellow precipitate formed was filtered off, reslurried in water and then dried over P₂O₅. 2.3 g of N-1-(2-dimethylaminoethyl)-2-methyl-4-nitro-1-aminobenzene (28) was obtained.

### Step 2: Synthesis of N-1-(2-dimethylaminoethyl)-2-methylbenzene-1,4-diamine dihydrochloride (29)

The N-1-(2-dimethylaminoethyl)-2-methyl-4-nitro-1-aminobenzene (28) obtained above was reduced with a boiling zinc/ammonium chloride/water/ethanol mixture. The corresponding amine was isolated in dihydrochloride form.

The proton NMR and mass spectra were in accordance with the expected structure of the product.

### Example 4

### Synthesis of 2-methyl-N-4-(2-pyrrolidin-1-ylethyl)benzene-1,4-diamine dihydrochloride (31)

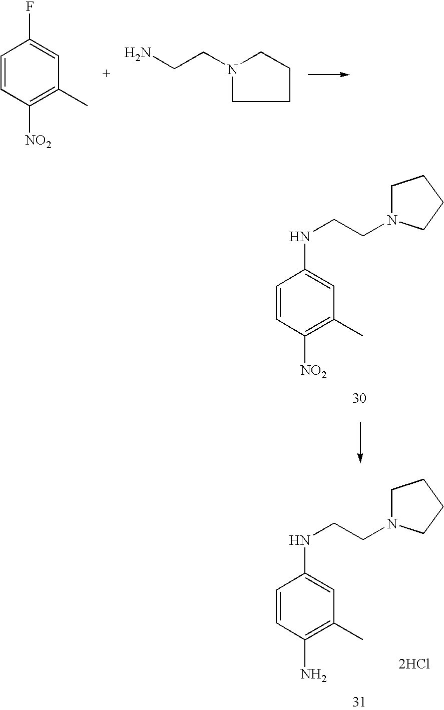

### Step 1: Synthesis of 2-methyl-N-4-(2-pyrrolidin-1-ylethyl)-4-nitro-1-aminobenzene (30)

2 g of 5-fluoro-2-nitrotoluene was added to a solution of 20 ml of N-methylpyrrolidinone, 1.76 g of 2-(1-pyrrolidino)ethylamine, and 1.57 g of triethylamine. The reaction medium was heated at 60° C. for 12 hours and, after cooling to room temperature, was then poured into a water and ice mixture. The resulting medium was extracted with ethyl acetate and the organic phase was then concentrated under reduced pressure. 3.2 g of 2-methyl-N-4-(2-pyrrolidin-1-ylethyl)-4-nitro-1-aminobenzene (30) was obtained.

### Step 2: Synthesis of 2-methyl-N-4-(2-pyrrolidin-1-ylethyl)benzene-1,4-diamine dihydrochloride (31)

The 2-methyl-N-4-(2-pyrrolidin-1-ylethyl)-4-nitro-1-aminobenzene (30) obtained above was reduced with a boiling zinc/ammonium chloride/water/ethanol mixture. The corresponding amine was isolated in dihydrochloride form.

The proton NMR and mass spectra were in accordance with the expected structure of the product.

### Example 5

### Synthesis of N-1-(3-diethylaminopropyl)-2-methylbenzene-1,4-diamine dihydrochloride (33)+

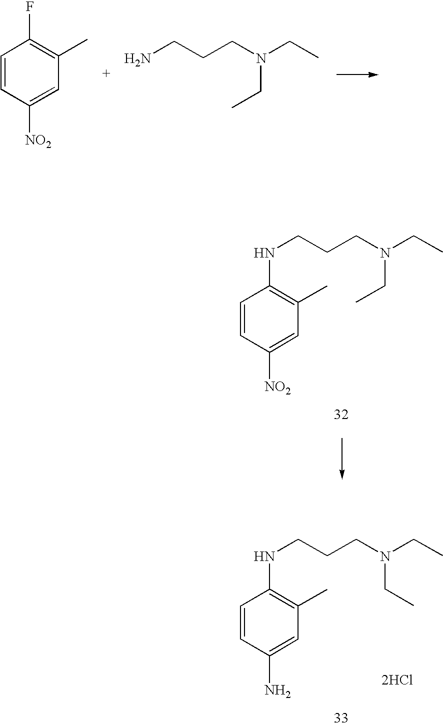

### Step 1: Synthesis of N-1-(3-diethylaminopropyl)-2-methyl-4-nitro-1-aminobenzene (32)

2 g of 2-fluoro-5-nitrotoluene was added to a solution of 20 ml of N-methylpyrrolidinone, 2.01 g of 3-diethylaminopropylamine, and 2.14 g of K₂CO₃. The reaction medium was heated at 60° C. for 12 hours and, after cooling to room temperature, was then poured into a water and ice mixture. The yellow precipitate formed was filtered off, reslurried in water and then dried over P₂O₅. 3.35 g of N-1-(3-diethylaminopropyl)-2-methyl-4-nitro-1-aminobenzene (32) was obtained.

### Step 2: Synthesis of N-1-(3-diethylaminopropyl)-2-methylbenzene-1,4-diamine dihydrochloride (33)

The N-1-(3-diethylaminopropyl)-2-methyl-4-nitro-1-aminobenzene (32) obtained above was reduced with a boiling zinc/ammonium chloride/water/ethanol mixture. The corresponding amine was isolated in dihydrochloride form.

The proton NMR and mass spectra were in accordance with the expected structure of the product.

### Example 6

### Synthesis of N-1-(3-dibutylaminopropyl)-2-methylbenzene-1,4-diamine dihydrochloride (35)

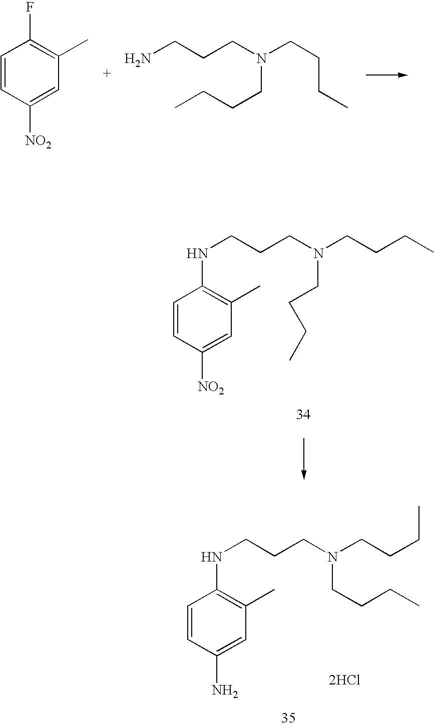

### Step 1: Synthesis of N-1-(3-dibutylaminopropyl)-2-methyl-4-nitro-1-aminobenzene (34)

2 g of 2-fluoro-5-nitrotoluene was added to a solution of 20 ml of N-methylpyrrolidinone, 2.88 g of 3-dibutylaminopropylamine, and 1.57 g of triethylamine. The reaction medium was heated at 60° C. for 15 hours and, after cooling to room temperature, was then poured into a water and ice mixture. The reaction medium was extracted with ethyl acetate and the organic phase was then concentrated under reduced pressure. 0.74 g of N-1-(3-dibutylaminopropyl)-2-methyl-4-nitro-1-aminobenzene (34) was obtained.

### Step 2: Synthesis of N-1-(3-dibutylaminopropyl)-2-methylbenzene-1,4-diamine dihydrochloride (35)

The N-1-(3-dibutylaminopropyl)-2-methyl-4-nitro-1-aminobenzene (34) obtained above was reduced with a boiling zinc/ammonium chloride/water/ethanol mixture. The corresponding amine was isolated in dihydrochloride form.

The proton NMR and mass spectra were in accordance with the expected structure of the product.

### Example 7

### Synthesis of N-1-(3-dimethylaminopropyl)-2-methylbenzene-1,4-diamine dihydrochloride (37)

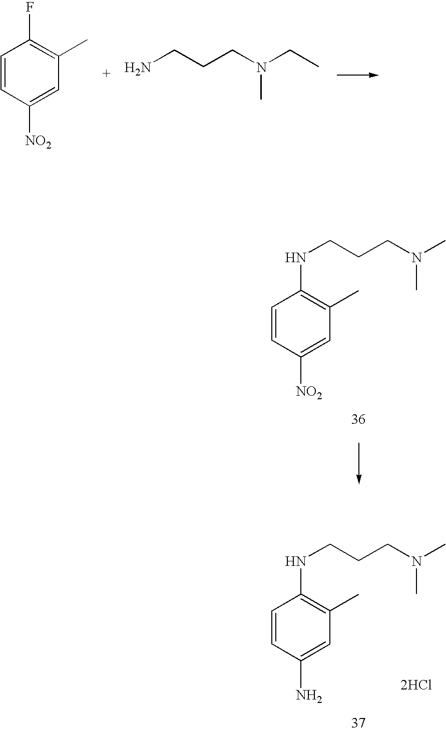

### Step 1: Synthesis of N-1-(3-dimethylaminopropyl)-2-methyl-4-nitro-1-aminobenzene (36)

2 g of 2-fluoro-5-nitrotoluene was added to a solution of 20 ml of N-methylpyrrolidinone, 1.58 g of N,N-dimethyl-1,3-diaminopropane, and 2.14 g of K₂CO₃. The reaction medium was heated at 60° C. for 12 hours and, after cooling to room temperature, was then poured into a water and ice mixture. The yellow precipitate formed was filtered off, reslurried in water and then dried over P₂O₅. 3 g of N-1-(3-dimethylaminopropyl)-2-methyl-4-nitro-1-aminobenzene (36) was obtained.

### Step 2: Synthesis of N-1-(3-dimethylaminopropyl)-2-methylbenzene-1,4-diamine dihydrochloride (37)

The N-1-(3-dimethylaminopropyl)-2-methyl-4-nitro-1-aminobenzene (36) obtained above was reduced with a boiling zinc/ammonium chloride/water/ethanol mixture. The corresponding amine was isolated in dihydrochloride form.

The proton NMR and mass spectra were in accordance with the expected structure of the product.

### Example 8

### Synthesis of N-1-(2-diisopropylaminoethyl)-2-methylbenzene-1,4-diamine dihydrochloride (39)+

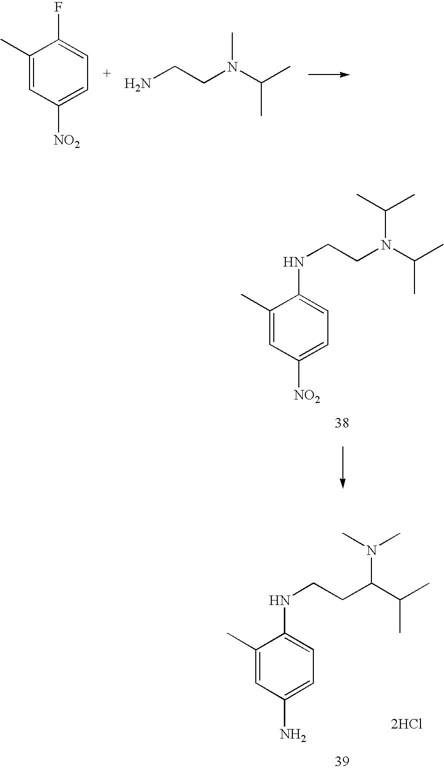

### Step 1: Synthesis of N-1-(2-diisopropylaminoethyl)-2-methyl-4-nitro-1-aminobenzene (38)

2 g of 2-fluoro-5-nitrotoluene was added to a solution of 20 ml of N-methylpyrrolidinone, 2.23 g of N,N-diisopropylethylenediamine, and 1.57 g of triethylamine. The reaction medium was heated at 60° C. for 12 hours and, after cooling to room temperature, was then poured into a water and ice mixture. The yellow precipitate formed was filtered off, reslurried in water and then dried over P₂O₅. 3.3 g of N-1-(2-diisopropylaminoethyl)-2-methyl-4-nitro-1-aminobenzene (38) was obtained.

### Step 2: Synthesis of N-1-(2-diisopropylaminoethyl)-2-methylbenzene-1,4-diamine dihydrochloride (39)

The N-1-(2-diisopropylaminoethyl)-2-methyl-4-nitro-1-aminobenzene (38) obtained above was reduced with a boiling zinc/ammonium chloride/water/ethanol mixture. The corresponding amine was isolated in dihydrochloride form.

The proton NMR and mass spectra were in accordance with the expected structure of the product.

### Example 9

### Synthesis of 2-methyl-N-1-(3-pyrrolidin-1-ylpropyl)benzene-1,4-diamine dihydrochloride (41)

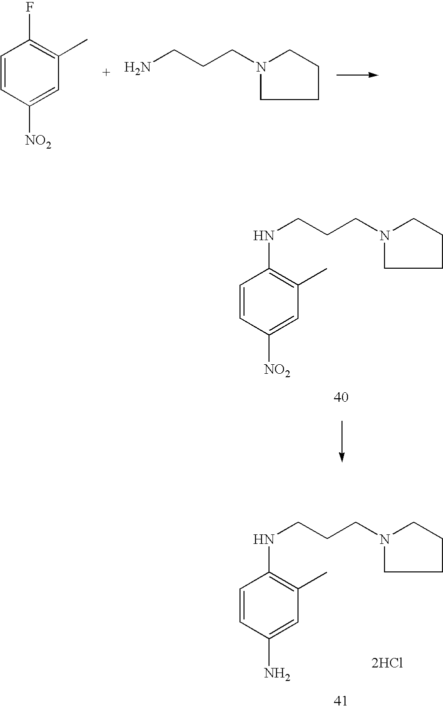

### Step 1: Synthesis of 2-methyl-N-1-(3-pyrrolidin-1-ylpropyl)-4-nitro-1-aminobenzene (40)

2 g of 2-fluoro-5-nitrotoluene was added to a solution of 20 ml of N-methylpyrrolidinone, 1.98 g of N-(3-aminopropyl)pyrrolidine, and 2.14 g of K₂CO₃. The reaction medium was heated at 60° C. for 15 hours and, after cooling to room temperature, was then poured into a water and ice mixture. The yellow precipitate formed was filtered off, reslurried in water and then dried over P₂O₅. 3.2 g of 2-methyl-N-1-(3-pyrrolidin-1-ylpropyl)-4-nitro-1-aminobenzene (40) was obtained.

### Step 2: Synthesis of 2-methyl-N-1-(3-pyrrolidin-1-ylpropyl)benzene-1,4-diamine dihydrochloride (41)

The 2-methyl-N-1-(3-pyrrolidin-1-ylpropyl)-4-nitro-1-aminobenzene (40) obtained above was reduced with a boiling zinc/ammonium chloride/water/ethanol mixture. The corresponding amine was isolated in dihydrochloride form.

The proton NMR and mass spectra were in accordance with the expected structure of the product.

### Example 10

### Synthesis of 3-methyl-N-1-(3-pyrrolidin-1-ylpropyl)benzene-1,4-diamine dihydrochloride (43)

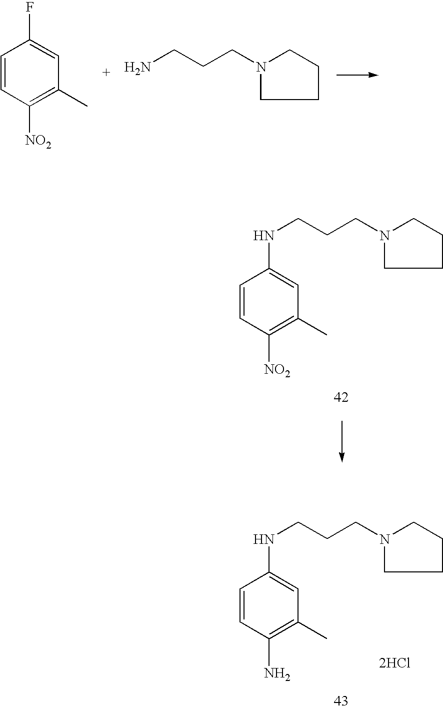

### Step 1: Synthesis of 3-methyl-N-1-(3-pyrrolidin-1-ylpropyl)-4-nitro-1-aminobenzene (42)

2 g of 5-fluoro-2-nitrotoluene was added to a solution of 20 ml of N-methylpyrrolidinone, 1.98 g of (N-3-aminopropyl)pyrrolidine, and 1.57 g of triethylamine. The reaction medium was heated at 60° C. for 12 hours and, after cooling to room temperature, was then poured into a water and ice mixture. The resulting medium was extracted with dichloromethane and the organic phase was then concentrated under reduced pressure. 3.39 g of 3-methyl-N-1-(3-pyrrolidin-1-ylpropyl)-4-nitro-1-aminobenzene (42) was obtained.

### Step 2: Synthesis of 3-methyl-N-1-(3-pyrrolidin-1-ylpropyl)benzene-1,4-diamine dihydrochloride

The 3-methyl-N-1-(3-pyrrolidin-1-ylpropyl)-4-nitro-1-aminobenzene (42) obtained above was reduced with a boiling zinc/ammonium chloride/water/ethanol mixture. The corresponding amine was isolated in dihydrochloride form.

The proton NMR and mass spectra were in accordance with the expected structure of the product.

### Example 11

### Synthesis of 1-[3-(4-amino-2-methylphenylamino)propyl]pyrrolidin-2-one dihydrochloride (45)

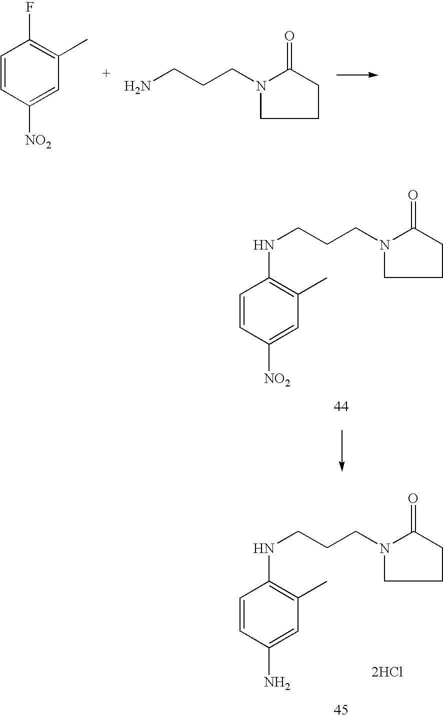

### Step 1: Synthesis of 1-[3-(4-nitro-3-methylphenylamino)propyl]pyrrolidin-2-one (44)

2 g of 2-fluoro-5-nitrotoluene was added to a solution of 20 ml of N-methylpyrrolidinone, 0.41 g of N-(3′aminopropyl)-2-pyrrolidinone, and 0.29 g of triethylamine. The reaction medium was heated at 60° C. for 15 hours and, after cooling to room temperature, was then poured into a water and ice mixture. The yellow precipitate formed was filtered off, reslurried in water and then dried over P₂O₅. 0.38 g of 1-[3-(4-nitro-3-methylphenylamino)propyl]pyrrolidin-2-one (44) was obtained.

### Step 2: Synthesis of 1-[3-(4-amino-3-methylphenylamino)propyl]pyrrolidin-2-one dihydrochloride (45)

The 1-[3-(4-nitro-3-methylphenylamino)propyl]pyrrolidin-2-one (44) obtained above was reduced with a boiling zinc/ammonium chloride/water/ethanol mixture. The corresponding amine was isolated in dihydrochloride form.

The proton NMR and mass spectra were in accordance with the expected structure of the product.

### Example 12

### Synthesis of 3-methyl-N-4-(2-pyrrolidin-1-ylethyl)benzene-1,4-diamine dihydrochloride (47)

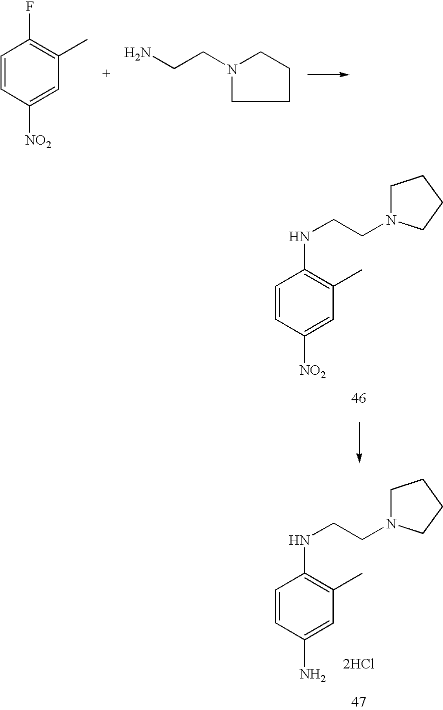

### Step 1: Synthesis of 3-methyl-N-4-(2-pyrrolidin-1-ylethyl)-4-nitro-1-aminobenzene (46)

2 g of 2-fluoro-5-nitrotoluene was added to a solution of 20 ml of N-methylpyrrolidinone, 1.76 g of N-(3-aminoethyl)pyrrolidine, and 1.56 g of triethylamine. The reaction medium was heated at 60° C. for 15 hours and, after cooling to room temperature, was then poured into a water and ice mixture. The yellow precipitate formed was filtered off, reslurried in water and then dried over P₂O₅. 2.8 g of 3-methyl-N-4-(2-pyrrolidin-1-ylethyl)-4-nitro-1-aminobenzene (46) was obtained.

### Step 2: Synthesis of 3-methyl-N-4-(2-pyrrolidin-1-ylethyl)benzene-1,4-diamine dihydrochloride (47)

The 3-methyl-N-4-(2-pyrrolidin-1-ylethyl)-4-nitro-1-aminobenzene (46) obtained above was reduced with a boiling zinc/ammonium chloride/water/ethanol mixture. The corresponding amine was isolated in dihydrochloride form.

The proton NMR and mass spectra were in accordance with the expected structure of the product.

### Example 13

### Synthesis of N-(2-aminoethyl)-N-(4-amino-3-methoxyphenyl)amine dihydrochloride (49)

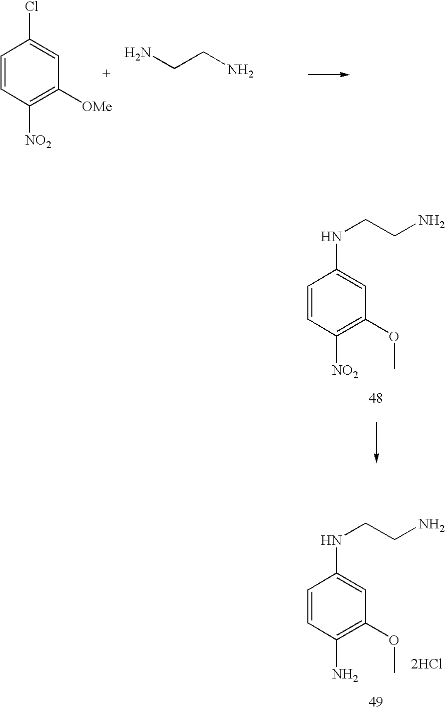

5-Chloro-2-nitroanisole (0.2 mmol in 500 μL of NMP-EtOH), ethylenediamine (0.24 mmol in 500 μL of NMP), and diisopropylethylamine (0.48 mmol) were heated at 110° C. for 24 hours. The reaction medium was then cooled and concentrated under vacuum. The residue was taken up in 2 mL of ethanol, and cyclohexene (1 mL) was added, along with a suspension of 50 mg of 5% palladium-on-charcoal in 500 μL of ethanol. This mixture was heated at 80° C. for 3 hours. The reaction medium was then filtered and treated with hydrochloric ethanol solution. The solution was then concentrated to give the desired compound in dihydrochloride form.

The proton NMR and mass spectra were in accordance with the expected structure of the product.

### Examples 14 to 24

### Dye Composition Using N-1-(2-aminoethyl)-2-methoxymethylbenzene-1,4-diamine trihydrochloride (25)

### Examples 14 to 20

### Dyeing in Acidic Medium

The following dye compositions were prepared:

| | Example | | | | | | | | | | | | | |
|---|---|---|---|---|---|---|---|---|---|---|---|---|---|---|
| | 14 | | 15 | | 16 | | 17 | | 18 | | 19 | | 20 | |
| N-1-(2-Aminoethyl)-2- | 10⁻³ | mol | 10⁻³ | mol | 10⁻³ | mol | 10⁻³ | mol | 10⁻³ | mol | 10⁻³ | mol | 10⁻³ | mol |
| methoxymethylbenzene-1,4- | | | | | | | | | | | | | | |
| diamine trihydrochloride (25) | | | | | | | | | | | | | | |
| Benzene-1,3-diol | 10⁻³ | mol | | | | | | | | | | | | |
| 5-Amino-2-methylphenol | | | 10⁻³ | mol | | | | | | | | | | |
| 1H-Indol-6-ol | | | | | 10⁻³ | mol | | | | | | | | |
| 2-Aminopyridin-3-ol | | | | | | | 10⁻³ | mol | | | | | | |
| 3,6-Dimethyl-1H-pyrazolo[5,1- | | | | | | | | | 10⁻³ | mol | | | | |
| c][1,2,4]triazole | | | | | | | | | | | | | | |
| 2-(2,4-Diaminophenoxy)- | | | | | | | | | | | 10⁻³ | mol | | |
| ethanol hydrochloride | | | | | | | | | | | | | | |
| 3-Amino-2-chloro-6- | | | | | | | | | | | | | 10⁻³ | mol |
| methylphenol hydrochloride | | | | | | | | | | | | | | |
| Dye support (1) | (*) | | (*) | | (*) | | (*) | | (*) | | (*) | | (*) | |
| Demineralized water qs | 100 | g | 100 | g | 100 | g | 100 | g | 100 | g | 100 | g | 100 | g |

(*): dye support (1) pH 7

| | | |
|---|---|---|
| 96° ethyl alcohol | 20.8 | g |
| Sodium metabisulfite as an aqueous 35% solution | 0.23 | g A.M. |
| Pentasodium salt of diethylenetriaminepentaacetic acid | 0.48 | g A.M. |
| as an aqueous 40% solution | | |
| C₈-C₁₀ Alkyl polyglucoside as an aqueous 60% solution | 3.6 | g A.M. |
| Benzyl alcohol | 2.0 | g |
| Polyethylene glycol containing 8 ethylene oxide units | 3.0 | g |
| Na₂HPO₄ | 0.28 | g |
| KH₂PO₄ | 0.46 | g |

At the time of use, each composition was mixed with an equal weight of 20-volume aqueous hydrogen peroxide solution (6% by weight). A final pH of 7 was obtained.

Each mixture obtained was applied to locks of grey hair containing 90% white hairs. After an action time of 30 minutes, the locks were rinsed, washed with a standard shampoo, rinsed again and then dried.

The shades obtained are given in the table below:

| | Example | | | | | | |
|---|---|---|---|---|---|---|---|
| | 14 | 15 | 16 | 17 | 18 | 19 | 20 |
| Shade | or- | orange | strong | orange | orange | strong | orange |
| ob- | ange | | orange | | | brown | |
| served | | | | | | | |

### Examples 21 to 24

### Dyeing in Basic Medium

The following dye compositions were prepared:

| | Example | | | | | | | |
|---|---|---|---|---|---|---|---|---|
| | 21 | | 22 | | 23 | | 24 | |
| N-1-(2-Aminoethyl)-2- | 10⁻³ | mol | 10⁻³ | mol | 10⁻³ | mol | 10⁻³ | mol |
| methoxymethylbenzene-1,4- | | | | | | | | |
| diamine trihydrochloride | | | | | | | | |
| (25) | | | | | | | | |
| 1H-Indol-6-ol | 10⁻³ | mol | | | | | | |
| 2-Aminopyridin-3-ol | | | 10⁻³ | mol | | | | |
| 2-(2,4-Diaminophenoxy)- | | | | | 10⁻³ | mol | | |
| ethanol hydrochloride | | | | | | | | |
| 3-Amino-2-chloro-6-methyl-phenol | | | | | | | 10⁻³ | mol |
| hydrochloride | | | | | | | | |
| Dye support (2) | (*) | | (*) | | (*) | | (*) | |
| Demineralized water qs | 100 | g | 100 | g | 100 | g | 100 | g |

(*): dye support (2) pH 9.5

| | | |
|---|---|---|
| 96° ethyl alcohol | 20.8 | g |
| Sodium metabisulfite as an aqueous 35% solution | 0.23 | g A.M. |
| Pentasodium salt of diethylenetriaminepentaacetic acid | 0.48 | g A.M. |
| as an aqueous 40% solution | | |
| C₈-C₁₀ Alkyl polyglucoside as an aqueous 60% solution | 3.6 | g A.M. |
| Benzyl alcohol | 2.0 | g |
| Polyethylene glycol containing 8 ethylene oxide units | 3.0 | g |
| NH₄Cl | 4.32 | g |
| Aqueous ammonia containing 20% NH₃ | 2.94 | g |

At the time of use, each composition was mixed with an equal weight of 20-volume aqueous hydrogen peroxide solution (6% by weight). A final pH of 9.5 was obtained.

Each mixture obtained was applied to locks of grey hair containing 90% white hairs. After an action time of 30 minutes, the locks were rinsed, washed with a standard shampoo, rinsed again and then dried.

The shades obtained are given in the table below:

Example

| | 21 | 22 | 23 | 24 |
|---|---|---|---|---|
| Shade observed | orange | orange | yellow | orange |

### Examples 25 to 33

### Dye Composition Using 1-[3-(4-amino-3-methylphenylamino)propyl]pyrrolidin-2-one dihydrochloride (27)

### Examples 25 to 29

### Dyeing in Acidic Medium

The following dye compositions were prepared:

| | Example | | | | | | | | | |
|---|---|---|---|---|---|---|---|---|---|---|
| | 25 | | 26 | | 27 | | 28 | | 29 | |
| 1-[3-(4-Amino-3- | 10⁻³ | mol | 10⁻³ | mol | 10⁻³ | mol | 10⁻³ | mol | 10⁻³ | mol |
| methylphenylamino)propyl]- | | | | | | | | | | |
| pyrrolidin-2-one | | | | | | | | | | |
| dihydrochloride (27) | | | | | | | | | | |
| 5-Amino-2-methylphenol | 10⁻³ | mol | | | | | | | | |
| 1H-Indol-6-ol | | | 10⁻³ | mol | | | | | | |
| 2-Aminopyridin-3-ol | | | | | 10⁻³ | mol | | | | |
| 2-(2,4- | | | | | | | 10⁻³ | mol | | |
| Diaminophenoxy)ethanol | | | | | | | | | | |
| hydrochloride | | | | | | | | | | |
| 3-Amino-2-chloro-6- | | | | | | | | | 10⁻³ | mol |
| methylphenol | | | | | | | | | | |
| hydrochloride | | | | | | | | | | |
| Dye support (1) | (*) | | (*) | | (*) | | (*) | | (*) | |
| Demineralized water qs | 100 | g | 100 | g | 100 | g | 100 | g | 100 | g |

(*): dye support (1) pH 7

| | | |
|---|---|---|
| 96° ethyl alcohol | 20.8 | g |
| Sodium metabisulfite as an aqueous 35% solution | 0.23 | g A.M. |
| Pentasodium salt of diethylenetriaminepentaacetic acid | 0.48 | g A.M. |
| as an aqueous 40% solution | | |
| C₈-C₁₀ Alkyl polyglucoside as an aqueous 60% solution | 3.6 | g A.M. |
| Benzyl alcohol | 2.0 | g |
| Polyethylene glycol containing 8 ethylene oxide units | 3.0 | g |
| Na₂HPO₄ | 0.28 | g |
| KH₂PO₄ | 0.46 | g |

At the time of use, each composition was mixed with an equal weight of 20-volume aqueous hydrogen peroxide solution (6% by weight). A final pH of 7 was obtained.

Each mixture obtained was applied to locks of grey hair containing 90% white hairs. After an action time of 30 minutes, the locks were rinsed, washed with a standard shampoo, rinsed again and then dried.

The shades obtained are given in the table below:

| | Example | | | | |
|---|---|---|---|---|---|
| | 25 | 26 | 27 | 28 | 29 |
| Shade observed | strong violet- | orange | orange- | strong | yellow |
| | grey | | brown | blue- | |
| | | | | green | |
| | | | | grey | |

### Examples 30 to 33

### Dyeing in Basic Medium

The following dye compositions were prepared:

| | Example | | | | | | | |
|---|---|---|---|---|---|---|---|---|
| | 30 | | 31 | | 32 | | 33 | |
| 1-[3-(4-Amino-3- | 10⁻³ | mol | 10⁻³ | mol | 10⁻³ | mol | 10⁻³ | mol |
| methylphenylamino) propyl]- | | | | | | | | |
| pyrrolidin-2-one | | | | | | | | |
| dihydrochloride (27) | | | | | | | | |
| 5-Amino-2- | 10⁻³ | mol | | | | | | |
| methylphenol | | | | | | | | |
| 3,6-Dimethyl-1H- | | | 10⁻³ | mol | | | | |
| pyrazolo[5,1-c]- | | | | | | | | |
| [1,2,4]triazole | | | | | | | | |
| 2-(2,4- | | | | | 10⁻³ | mol | | |
| Diaminophenoxy)ethanol | | | | | | | | |
| hydrochloride | | | | | | | | |
| 3-Amino-2-chloro-6- | | | | | | | 10⁻³ | mol |
| methylphenol | | | | | | | | |
| hydrochloride | | | | | | | | |
| Dye support (2) | (*) | | (*) | | (*) | | (*) | |
| Demineralized water | 100 | g | 100 | g | 100 | g | 100 | g |
| qs | | | | | | | | |

(*): dye support (2) pH 9.5

| | | |
|---|---|---|
| 96° ethyl alcohol | 20.8 | g |
| Sodium metabisulfite as an aqueous 35% solution | 0.23 | g A.M. |
| Pentasodium salt of diethylenetriaminepentaacetic acid | 0.48 | g A.M. |
| as an aqueous 40% solution | | |
| C₈-C₁₀ Alkyl polyglucoside as an aqueous 60% solution | 3.6 | g A.M. |
| Benzyl alcohol | 2.0 | g |
| Polyethylene glycol containing 8 ethylene oxide units | 3.0 | g |
| NH₄Cl | 4.32 | g |
| Aqueous ammonia containing 20% NH₃ | 2.94 | g |

At the time of use, each composition was mixed with an equal weight of 20-volume aqueous hydrogen peroxide solution (6% by weight). A final pH of 9.5 was obtained.

Each mixture obtained was applied to locks of grey hair containing 90% white hairs. After an action time of 30 minutes, the locks were rinsed, washed with a standard shampoo, rinsed again and then dried.

The shades obtained are given in the table below:

Example

| | 30 | 31 | 32 | 33 |
|---|---|---|---|---|
| Shade observed | violet-grey | chromatic | blue- | strong |
| | | red | green | blue |

### Examples 34 to 45

### Dye Composition Using 2-methyl-N-1-(3-pyrrolidin-1-ylpropyl)benzene-1,4-diamine dihydrochloride (41)

### Examples 34 to 40

### Dyeing in Acidic Medium

The following dye compositions were prepared:

| | Example | | | | | | | | | | | | | |
|---|---|---|---|---|---|---|---|---|---|---|---|---|---|---|
| | 34 | | 35 | | 36 | | 37 | | 38 | | 39 | | 40 | |
| 2-Methyl-N-1-(3- | 10⁻³ | mol | 10⁻³ | mol | 10⁻³ | mol | 10⁻³ | mol | 10⁻³ | mol | 10⁻³ | mol | 10⁻³ | mol |
| pyrrolidin-1- | | | | | | | | | | | | | | |
| ylpropyl)benzene-1,4- | | | | | | | | | | | | | | |
| diamine dihydrochloride | | | | | | | | | | | | | | |
| (41) | | | | | | | | | | | | | | |
| Benzene-1,3-diol | 10⁻³ | mol | | | | | | | | | | | | |
| 5-Amino-2-methylphenol | | | 10⁻³ | mol | | | | | | | | | | |
| 1H-Indol-6-ol | | | | | 10⁻³ | mol | | | | | | | | |
| 2-Aminopyridin-3-ol | | | | | | | 10⁻³ | mol | | | | | | |
| 3,6-Dimethyl-1H- | | | | | | | | | 10⁻³ | mol | | | | |
| pyrazolo-[5,1-c]- | | | | | | | | | | | | | | |
| [1,2,4]triazole | | | | | | | | | | | | | | |
| 2-(2,4- | | | | | | | | | | | 10⁻³ | mol | | |
| Diaminophenoxy)ethanol | | | | | | | | | | | | | | |
| hydrochloride | | | | | | | | | | | | | | |
| 3-Amino-2-chloro-6- | | | | | | | | | | | | | 10⁻³ | mol |
| methylphenol | | | | | | | | | | | | | | |
| hydrochloride | | | | | | | | | | | | | | |
| Dye support (1) | (*) | | (*) | | (*) | | (*) | | (*) | | (*) | | (*) | |
| Demineralized water qs | 100 | g | 100 | g | 100 | g | 100 | g | 100 | g | 100 | g | 100 | g |

(*): dye support (1) pH 7

| | | |
|---|---|---|
| 96° ethyl alcohol | 20.8 | g |
| Sodium metabisulfite as an aqueous 35% solution | 0.23 | g A.M. |
| Pentasodium salt of diethylenetriaminepentaacetic acid | 0.48 | g A.M. |
| as an aqueous 40% solution | | |
| C₈-C₁₀ Alkyl polyglucoside as an aqueous 60% solution | 3.6 | g A.M. |
| Benzyl alcohol | 2.0 | g |
| Polyethylene glycol containing 8 ethylene oxide units | 3.0 | g |
| Na₂HPO₄ | 0.28 | g |
| KH₂PO₄ | 0.46 | g |

At the time of use, each composition was mixed with an equal weight of 20-volume aqueous hydrogen peroxide solution (6% by weight). A final pH of 7 was obtained.

Each mixture obtained was applied to locks of grey hair containing 90% white hairs. After an action time of 30 minutes, the locks were rinsed, washed with a standard shampoo, rinsed again and then dried.

The shades obtained are given in the table below:

| | Example | | | | | | |
|---|---|---|---|---|---|---|---|
| | 34 | 35 | 36 | 37 | 38 | 39 | 40 |
| Shade observed | yellow-brown | strong | brown | red- | orange | strong | strong |
| | | violet-grey | | brown | | blue- | blue- |
| | | | | | | grey | violet |

### Examples 41 to 45

### Dyeing in Basic Medium

The following dye compositions were prepared:

| | Example | | | | | | | | | |
|---|---|---|---|---|---|---|---|---|---|---|
| | 41 | | 42 | | 43 | | 44 | | 45 | |
| 2-Methyl-N-1-(3-pyrrolidin- | 10⁻³ | mol | 10⁻³ | mol | 10⁻³ | mol | 10⁻³ | mol | 10⁻³ | mol |
| 1-ylpropyl)- | | | | | | | | | | |
| benzene-1,4-diamine | | | | | | | | | | |
| dihydrochloride (41) | | | | | | | | | | |
| 5-Amino-2- | 10⁻³ | mol | | | | | | | | |
| methylphenol | | | | | | | | | | |
| 2-Aminopyridin-3-ol | | | 10⁻³ | mol | | | | | | |
| 3,6-Dimethyl-1H- | | | | | 10⁻³ | mol | | | | |
| pyrazolo[5,1-c]- | | | | | | | | | | |
| [1,2,4]triazole | | | | | | | | | | |
| 2-(2,4- | | | | | | | 10⁻³ | mol | | |
| Diaminophenoxy)ethanol | | | | | | | | | | |
| hydrochloride | | | | | | | | | | |
| 3-Amino-2-chloro-6- | | | | | | | | | 10⁻³ | mol |
| methylphenol | | | | | | | | | | |
| hydrochloride | | | | | | | | | | |
| Dye support (2) | (*) | | (*) | | (*) | | (*) | | (*) | |
| Demineralized water | 100 | g | 100 | g | 100 | g | 100 | g | 100 | g |
| qs | | | | | | | | | | |

(*): dye support (2) pH 9.5

| | | |
|---|---|---|
| 96° ethyl alcohol | 20.8 | g |
| Sodium metabisulfite as an aqueous 35% solution | 0.23 | g A.M. |
| Pentasodium salt of diethylenetriaminepentaacetic acid | 0.48 | g A.M. |
| as an aqueous 40% solution | | |
| C₈-C₁₀ Alkyl polyglucoside as an aqueous 60% solution | 3.6 | g A.M. |
| Benzyl alcohol | 2.0 | g |
| Polyethylene glycol containing 8 ethylene oxide units | 3.0 | g |
| NH₄Cl | 4.32 | g |
| Aqueous ammonia containing 20% NH₃ | 2.94 | g |

At the time of use, each composition was mixed with an equal weight of 20-volume aqueous hydrogen peroxide solution (6% by weight). A final pH of 9.5 was obtained.

Each mixture obtained was applied to locks of grey hair containing 90% white hairs. After an action time of 30 minutes, the locks were rinsed, washed with a standard shampoo, rinsed again and then dried.

The shades obtained were given in the table below:

| | Example | | | | |
|---|---|---|---|---|---|
| | 41 | 42 | 43 | 44 | 45 |
| Shade observed | strong | orange | chromatic | strong | strong |
| | red-violet | | red | blue | blue- |
| | | | | | violet |

### Examples 46 to 53

### Dye Composition Using N-1-(3-diethylaminopropyl)-2-methylbenzene-1,4-diamine dihydrochloride (33)

### Examples 46 to 50

### Dyeing in Acidic Medium

The following dye compositions were prepared:

| | Example | | | | | | | | | |
|---|---|---|---|---|---|---|---|---|---|---|
| | 46 | | 47 | | 48 | | 49 | | 50 | |
| N-1-(3- | 10⁻³ | mol | 10⁻³ | mol | 10⁻³ | mol | 10⁻³ | mol | 10⁻³ | mol |
| Diethylaminopropyl)-2- | | | | | | | | | | |
| methylbenzene-1,4- | | | | | | | | | | |
| diamine dihydrochloride | | | | | | | | | | |
| (33) | | | | | | | | | | |
| 5-Amino-2-methylphenol | 10⁻³ | mol | | | | | | | | |
| 1H-Indol-6-ol | | | 10⁻³ | mol | | | | | | |
| 2-Aminopyridin-3-ol | | | | | 10⁻³ | mol | | | | |
| 2-(2,4- | | | | | | | 10⁻³ | mol | | |
| Diaminophenoxy)ethanol | | | | | | | | | | |
| hydrochloride | | | | | | | | | | |
| 3-Amino-2-chloro-6- | | | | | | | | | 10⁻³ | mol |
| methylphenol | | | | | | | | | | |
| hydrochloride | | | | | | | | | | |
| Dye support (1) | (*) | | (*) | | (*) | | (*) | | (*) | |
| Demineralized water qs | 100 | g | 100 | g | 100 | g | 100 | g | 100 | g |

(*): dye support (1) pH 7

| | | |
|---|---|---|
| 96° ethyl alcohol | 20.8 | g |
| Sodium metabisulfite as an aqueous 35% solution | 0.23 | g A.M. |
| Pentasodium salt of diethylenetriaminepentaacetic acid | 0.48 | g A.M. |
| as an aqueous 40% solution | | |
| C₈-C₁₀ Alkyl polyglucoside as an aqueous 60% solution | 3.6 | g A.M. |
| Benzyl alcohol | 2.0 | g |
| Polyethylene glycol containing 8 ethylene oxide units | 3.0 | g |
| Na₂HPO₄ | 0.28 | g |
| KH₂PO₄ | 0.46 | g |

At the time of use, each composition was mixed with an equal weight of 20-volume aqueous hydrogen peroxide solution (6% by weight). A final pH of 7 was obtained.

Each mixture obtained was applied to locks of grey hair containing 90% white hairs. After an action time of 30 minutes, the locks were rinsed, washed with a standard shampoo, rinsed again and then dried.

The shades obtained are given in the table below:

| | Example | | | | |
|---|---|---|---|---|---|
| | 46 | 47 | 48 | 49 | 50 |
| Shade observed | red-violet | brown | orange- | strong | strong |
| | grey | | brown | blue- | blue-violet |
| | | | | green | |
| | | | | grey | |

### Examples 50 to 53

### Dyeing in Basic Medium

The following dye compositions were prepared:

| | Example | | | | | | | |
|---|---|---|---|---|---|---|---|---|
| | 50 | | 51 | | 52 | | 53 | |
| N-1-(3- | 10⁻³ | mol | 10⁻³ | mol | 10⁻³ | mol | 10⁻³ | mol |
| Diethylamino- | | | | | | | | |
| propyl)-2- | | | | | | | | |
| methyl- | | | | | | | | |
| benzene-1,4- | | | | | | | | |
| diamine | | | | | | | | |
| dihydrochloride | | | | | | | | |
| (33) | | | | | | | | |
| 5-Amino-2- | 10⁻³ | mol | | | | | | |
| methylphenol | | | | | | | | |
| 3,6-Dimethyl- | | | 10⁻³ | mol | | | | |
| 1H- | | | | | | | | |
| pyrazolo | | | | | | | | |
| [5,1-c]- | | | | | | | | |
| [1,2,4]triazole | | | | | | | | |
| 2-(2,4- | | | | | 10⁻³ | mol | | |
| Diamino- | | | | | | | | |
| phenoxy) | | | | | | | | |
| ethanol | | | | | | | | |
| hydrochloride | | | | | | | | |
| 3-Amino-2- | | | | | | | 10⁻³ | mol |
| chloro-6- | | | | | | | | |
| methylphenol | | | | | | | | |
| hydrochloride | | | | | | | | |
| Dye support(2) | (*) | | (*) | | (*) | | (*) | |
| Demineralized | 100 | g | 100 | g | 100 | g | 100 | g |
| water qs | | | | | | | | |

(*): dye support (2) pH 9.5

| | | |
|---|---|---|
| 96° ethyl alcohol | 20.8 | g |
| Sodium metabisulfite as an aqueous 35% solution | 0.23 | g A.M. |
| Pentasodium salt of diethylenetriaminepentaacetic acid | 0.48 | g A.M. |
| as an aqueous 40% solution | | |
| C₈-C₁₀ Alkyl polyglucoside as an aqueous 60% solution | 3.6 | g A.M. |
| Benzyl alcohol | 2.0 | g |
| Polyethylene glycol containing 8 ethylene oxide units | 3.0 | g |
| NH₄Cl | 4.32 | g |
| Aqueous ammonia containing 20% NH₃ | 2.94 | g |

At the time of use, each composition was mixed with an equal weight of 20-volume aqueous hydrogen peroxide solution (6% by weight). A final pH of 9.5 was obtained.

Each mixture obtained was applied to locks of grey hair containing 90% white hairs. After an action time of 30 minutes, the locks were rinsed, washed with a standard shampoo, rinsed again and then dried.

The shades obtained are given in the table below:

| | Example | | | |
|---|---|---|---|---|
| | 50 | 51 | 52 | 53 |
| Shade observed | red-violet | chromatic | blue | strong blue- |
| | | red | | violet |

### Examples 54 to 60

### Dye Composition Using N-1-(2-dimethylaminoethyl)-2-methylbenzene-1,4-diamine dihydrochloride (29)

### Examples 54 to 56

### Dyeing in Acidic Medium

The following dye compositions were prepared:

| | Example | | | | | |
|---|---|---|---|---|---|---|
| | 54 | | 55 | | 56 | |
| N-1-(2- | 10⁻³ | mol | 10⁻³ | mol | 10⁻³ | mol |
| Dimethylaminoethyl)-2- | | | | | | |
| methylbenzene-1,4- | | | | | | |
| diamine dihydrochloride | | | | | | |
| (29) | | | | | | |
| 1H-Indol-6-ol | 10⁻³ | mol | | | | |
| 2-(2,4-Diaminophenoxy)- | | | 10⁻³ | mol | | |
| ethanol hydrochloride | | | | | | |
| 3-Amino-2-chloro-6- | | | | | 10⁻³ | mol |
| methylphenol | | | | | | |
| hydrochloride | | | | | | |
| Dye support(1) | (*) | | (*) | | (*) | |
| Demineralized water qs | 100 | g | 100 | g | 100 | g |

(*): dye support (1) pH 7

| | | |
|---|---|---|
| 96° ethyl alcohol | 20.8 | g |
| Sodium metabisulfite as an aqueous 35% solution | 0.23 | g A.M. |
| Pentasodium salt of diethylenetriaminepentaacetic acid | 0.48 | g A.M. |
| as an aqueous 40% solution | | |
| C₈-C₁₀ Alkyl polyglucoside as an aqueous 60% solution | 3.6 | g A.M. |
| Benzyl alcohol | 2.0 | g |
| Polyethylene glycol containing 8 ethylene oxide units | 3.0 | g |
| Na₂HPO₄ | 0.28 | g |
| KH₂PO₄ | 0.46 | g |

At the time of use, each composition was mixed with an equal weight of 20-volume aqueous hydrogen peroxide solution (6% by weight). A final pH of 7 was obtained.

Each mixture obtained was applied to locks of grey hair containing 90% white hairs. After an action time of 30 minutes, the locks were rinsed, washed with a standard shampoo, rinsed again and then dried.

The shades obtained are given in the table below:

Example

| | 54 | 55 | 56 |
|---|---|---|---|
| Shade observed | red-brown | strong | red-brown |
| | | blue-grey | |

### Examples 57 to 60

### Dyeing in Basic Medium

The following dye compositions were prepared:

| | Example | | | | | | | |
|---|---|---|---|---|---|---|---|---|
| | 57 | | 58 | | 59 | | 60 | |
| N-1-(2-Dimethylaminoethyl)- | 10⁻³ | mol | 10⁻³ | mol | 10⁻³ | mol | 10⁻³ | mol |
| 2-methylbenzene-1,4- | | | | | | | | |
| diamine | | | | | | | | |
| dihydrochloride (29) | | | | | | | | |
| 5-Amino-2-methyl- | 10⁻³ | mol | | | | | | |
| phenol | | | | | | | | |
| 3,6-Dimethyl-1H- | | | 10⁻³ | mol | | | | |
| pyrazolo[5,1-c]- | | | | | | | | |
| [1,2,4]triazole | | | | | | | | |
| 2-(2,4-Diaminophenoxy) | | | | | 10⁻³ | mol | | |
| ethanol | | | | | | | | |
| hydrochloride | | | | | | | | |
| 3-Amino-2-chloro-6- | | | | | | | 10⁻³ | mol |
| methylphenol | | | | | | | | |
| hydrochloride | | | | | | | | |
| Dye support (2) | (*) | | (*) | | (*) | | (*) | |
| Demineralized water | 100 | g | 100 | g | 100 | g | 100 | g |
| qs | | | | | | | | |

(*): dye support (2) pH 9.5

| | | |
|---|---|---|
| 96° ethyl alcohol | 20.8 | g |
| Sodium metabisulfite as an aqueous 35% solution | 0.23 | g A.M. |
| Pentasodium salt of diethylenetriaminepentaacetic | 0.48 | g A.M. |
| acid as an aqueous 40% solution | | |
| C₈-C₁₀ Alkyl polyglucoside as an aqueous 60% solution | 3.6 | g A.M. |
| Benzyl alcohol | 2.0 | g |
| Polyethylene glycol containing 8 ethylene oxide units | 3.0 | g |
| NH₄Cl | 4.32 | g |
| Aqueous ammonia containing 20% NH₃ | 2.94 | g |

At the time of use, each composition was mixed with an equal weight of 20-volume aqueous hydrogen peroxide solution (6% by weight). A final pH of 9.5 was obtained.

Each mixture obtained was applied to locks of grey hair containing 90% white hairs. After an action time of 30 minutes, the locks were rinsed, washed with a standard shampoo, rinsed again and then dried.

The shades obtained are given in the table below:

Example

| | 57 | 58 | 59 | 60 |
|---|---|---|---|---|
| Shade observed | red | chromatic red | strong | strong |
| | | | blue | violet |

### Examples 61 to 69

### Dye Composition Using N-1-(3-dimethylaminopropyl)-2-methylbenzene-1,4-diamine dihydrochloride (37)

### Examples 61 to 65

### Dyeing in Acidic Medium

The following dye compositions were prepared:

| | Example | | | | | | | | | |
|---|---|---|---|---|---|---|---|---|---|---|
| | 61 | | 62 | | 63 | | 64 | | 65 | |
| N-1-(3-Dimethylaminopropyl)- | 10⁻³ | mol | 10⁻³ | mol | 10⁻³ | mol | 10⁻³ | mol | 10⁻³ | mol |
| 2-methylbenzene- | | | | | | | | | | |
| 1,4-diamine | | | | | | | | | | |
| dihydrochloride (37) | | | | | | | | | | |
| 5-Amino-2-methylphenol | 10⁻³ | mol | | | | | | | | |
| 1H-Indol-6-ol | | | 10⁻³ | mol | | | | | | |
| 2-Aminopyridin-3-ol | | | | | 10⁻³ | mol | | | | |
| 2-(2,4- | | | | | | | 10⁻³ | mol | | |
| Diaminophenoxy)ethanol | | | | | | | | | | |
| hydrochloride | | | | | | | | | | |
| 3-Amino-2-chloro-6- | | | | | | | | | 10⁻³ | mol |
| methylphenol | | | | | | | | | | |
| hydrochloride | | | | | | | | | | |
| Dye support (1) | (*) | | (*) | | (*) | | (*) | | (*) | |
| Demineralized water qs | 100 | g | 100 | g | 100 | g | 100 | g | 100 | g |

(*): dye support (1) pH 7

| | | |
|---|---|---|
| 96° ethyl alcohol | 20.8 | g |
| Sodium metabisulfite as an aqueous 35% solution | 0.23 | g A.M. |
| Pentasodium salt of diethylenetriaminepentaacetic acid | 0.48 | g A.M. |
| as an aqueous 40% solution | | |
| C₈-C₁₀ Alkyl polyglucoside as an aqueous 60% solution | 3.6 | g A.M. |
| Benzyl alcohol | 2.0 | g |
| Polyethylene glycol containing 8 ethylene oxide units | 3.0 | g |
| Na₂HPO₄ | 0.28 | g |
| KH₂PO₄ | 0.46 | g |

At the time of use, each composition was mixed with an equal weight of 20-volume aqueous hydrogen peroxide solution (6% by weight). A final pH of 7 was obtained.

Each mixture obtained was applied to locks of grey hair containing 90% white hairs. After an action time of 30 minutes, the locks were rinsed, washed with a standard shampoo, rinsed again and then dried.

The shades obtained are given in the table below:

| | Example | | | | |
|---|---|---|---|---|---|
| | 61 | 62 | 63 | 64 | 65 |
| Shade observed | red-violet | brown | red-brown | strong | strong |
| | grey | | | blue-grey | blue-violet |

### Examples 66 to 69

### Dyeing in Basic Medium

The following dye compositions were prepared:

| | Example | | | | | | | |
|---|---|---|---|---|---|---|---|---|
| | 66 | | 67 | | 68 | | 69 | |
| N-1-(3-Dimethylaminopropyl)- | 10⁻³ | mol | 10⁻³ | mol | 10⁻³ | mol | 10⁻³ | mol |
| 2-methylbenzene-1,4- | | | | | | | | |
| diamine dihydrochloride | | | | | | | | |
| (37) | | | | | | | | |
| 5-Amino-2-methylphenol | 10⁻³ | mol | | | | | | |
| 3,6-Dimethyl-1H- | | | 10⁻³ | mol | | | | |
| pyrazolo[5,1-c]- | | | | | | | | |
| [1,2,4]triazole | | | | | | | | |
| 2-(2,4- | | | | | 10⁻³ | mol | | |
| Diaminophenoxy)- | | | | | | | | |
| ethanol hydrochloride | | | | | | | | |
| 3-Amino-2-chloro-6- | | | | | | | 10⁻³ | mol |
| methylphenol | | | | | | | | |
| hydrochloride | | | | | | | | |
| Dye support (2) | (*) | | (*) | | (*) | | (*) | |
| Demineralized water qs | 100 | g | 100 | g | 100 | g | 100 | g |

(*): dye support (2) pH 9.5

| | | |
|---|---|---|
| 96° ethyl alcohol | 20.8 | g |
| Sodium metabisulfite as an aqueous 35% solution | 0.23 | g A.M. |
| Pentasodium salt of diethylenetriaminepentaacetic | 0.48 | g A.M. |
| acid as an aqueous 40% solution | | |
| C₈-C₁₀ Alkyl polyglucoside as an aqueous 60% solution | 3.6 | g A.M. |
| Benzyl alcohol | 2.0 | g |
| Polyethylene glycol containing 8 ethylene oxide units | 3.0 | g |
| NH₄Cl | 4.32 | g |
| Aqueous ammonia containing 20% NH₃ | 2.94 | g |

At the time of use, each composition was mixed with an equal weight of 20-volume aqueous hydrogen peroxide solution (6% by weight). A final pH of 9.5 was obtained.

Each mixture obtained was applied to locks of grey hair containing 90% white hairs. After an action time of 30 minutes, the locks were rinsed, washed with a standard shampoo, rinsed again and then dried.

The shades obtained are given in the table below:

| | Example | | | |
|---|---|---|---|---|
| | 66 | 67 | 68 | 69 |
| Shade observed | red-violet | chromatic | strong blue | strong blue- |
| | | red | | violet |

### Examples 70 to 83

### Dye Composition Using N-1-(3-dibutylaminopropyl)-2-methylbenzene-1,4-diamine dihydrochloride (35)

### Examples 70 to 76

### Dyeing in Acidic Medium

The following dye compositions were prepared:

| | Example | | | | | | | | | | | | | |
|---|---|---|---|---|---|---|---|---|---|---|---|---|---|---|
| | 70 | | 71 | | 72 | | 73 | | 74 | | 75 | | 76 | |
| N-1-(3- | 10⁻³ | mol | 10⁻³ | mol | 10⁻³ | mol | 10⁻³ | mol | 10⁻³ | mol | 10⁻³ | mol | 10⁻³ | mol |
| Dibutylaminopropyl)-2- | | | | | | | | | | | | | | |
| methylbenzene-1,4- | | | | | | | | | | | | | | |
| diamine dihydrochloride | | | | | | | | | | | | | | |
| (35) | | | | | | | | | | | | | | |
| Benzene-1,3-diol | 10⁻³ | mol | | | | | | | | | | | | |
| 5-Amino-2-methylphenol | | | 10⁻³ | mol | | | | | | | | | | |
| 1H-Indol-6-ol | | | | | 10⁻³ | mol | | | | | | | | |
| 2-Aminopyridin-3-ol | | | | | | | 10⁻³ | mol | | | | | | |
| 3,6-Dimethyl-1H- | | | | | | | | | 10⁻³ | mol | | | | |
| pyrazolo[5,1-c]- | | | | | | | | | | | | | | |
| [1,2,4]triazole | | | | | | | | | | | | | | |
| 2-(2,4- | | | | | | | | | | | 10⁻³ | mol | | |
| Diaminophenoxy)ethanol | | | | | | | | | | | | | | |
| hydrochloride | | | | | | | | | | | | | | |
| 3-Amino-2-chloro-6- | | | | | | | | | | | | | 10⁻³ | mol |
| methylphenol | | | | | | | | | | | | | | |
| hydrochloride | | | | | | | | | | | | | | |
| Dye support (1) | (*) | | (*) | | (*) | | (*) | | (*) | | (*) | | (*) | |
| Demineralized water qs | 100 | g | 100 | g | 100 | g | 100 | g | 100 | g | 100 | g | 100 | g |

(*): dye support (1) pH 7

| | | |
|---|---|---|
| 96° ethyl alcohol | 20.8 | g |
| Sodium metabisulfite as an aqueous 35% solution | 0.23 | g A.M. |
| Pentasodium salt of diethylenetriaminepentaacetic acid | 0.48 | g A.M. |
| as an aqueous 40% solution | | |
| C₈-C₁₀ Alkyl polyglucoside as an aqueous 60% solution | 3.6 | g A.M. |
| Benzyl alcohol | 2.0 | g |
| Polyethylene glycol containing 8 ethylene oxide units | 3.0 | g |
| Na₂HPO₄ | 0.28 | g |
| KH₂PO₄ | 0.46 | g |

At the time of use, each composition was mixed with an equal weight of 20-volume aqueous hydrogen peroxide solution (6% by weight). A final pH of 7 was obtained.

Each mixture obtained was applied to locks of grey hair containing 90% white hairs. After an action time of 30 minutes, the locks were rinsed, washed with a standard shampoo, rinsed again and then dried.

The shades obtained are given in the table below:

| | Example | | | | | | |
|---|---|---|---|---|---|---|---|
| | 70 | 71 | 72 | 73 | 74 | 75 | 76 |
| Shade observed | brown | strong | strong | strong | orange- | strong | strong |
| | | violet | red-brown | red-brown | brown | blue | violet |

### Examples 77 to 83

### Dyeing in Basic Medium

The following dye compositions were prepared:

| | Example | | | | | | | | | | | | | |
|---|---|---|---|---|---|---|---|---|---|---|---|---|---|---|
| | 77 | | 78 | | 79 | | 80 | | 81 | | 82 | | 83 | |
| N-1-(3-Dibutylaminopropyl)- | 10⁻³ | mol | 10⁻³ | mol | 10⁻³ | mol | 10⁻³ | mol | 10⁻³ | mol | 10⁻³ | mol | 10⁻³ | mol |
| 2-methylbenzene-1,4- | | | | | | | | | | | | | | |
| diamine | | | | | | | | | | | | | | |
| dihydrochloride (35) | | | | | | | | | | | | | | |
| Benzene-1,3-diol | 10⁻³ | mol | | | | | | | | | | | | |
| 5-Amino-2- | | | 10⁻³ | mol | | | | | | | | | | |
| methylphenol | | | | | | | | | | | | | | |
| 1H-Indol-6-ol | | | | | 10⁻³ | mol | | | | | | | | |
| 2-Aminopyridin-3-ol | | | | | | | 10⁻³ | mol | | | | | | |
| 3,6-Dimethyl-1H- | | | | | | | | | 10⁻³ | mol | | | | |
| pyrazolo[5,1-c]- | | | | | | | | | | | | | | |
| [1,2,4]triazole | | | | | | | | | | | | | | |
| 2-(2,4- | | | | | | | | | | | 10⁻³ | mol | | |
| Diaminophenoxy)- | | | | | | | | | | | | | | |
| ethanol hydrochloride | | | | | | | | | | | | | | |
| 3-Amino-2-chloro-6- | | | | | | | | | | | | | 10⁻³ | mol |
| methylphenol | | | | | | | | | | | | | | |
| hydrochloride | | | | | | | | | | | | | | |
| Dye support (2) | (*) | | (*) | | (*) | | (*) | | (*) | | (*) | | (*) | |
| Demineralized water | 100 | g | 100 | g | 100 | g | 100 | g | 100 | g | 100 | g | 100 | g |
| qs | | | | | | | | | | | | | | |

(*): dye support (2) pH 9.5

| | | |
|---|---|---|
| 96° ethyl alcohol | 20.8 | g |
| Sodium metabisulfite as an aqueous 35% solution | 0.23 | g A.M. |
| Pentasodium salt of diethylenetriaminepentaacetic | 0.48 | g A.M. |
| acid as an aqueous 40% solution | | |
| C₈-C₁₀ Alkyl polyglucoside as an aqueous 60% solution | 3.6 | g A.M. |
| Benzyl alcohol | 2.0 | g |
| Polyethylene glycol containing 8 ethylene oxide units | 3.0 | g |
| NH₄Cl | 4.32 | g |
| Aqueous ammonia containing 20% NH₃ | 2.94 | g |

At the time of use, each composition was mixed with an equal weight of 20-volume aqueous hydrogen peroxide solution (6% by weight). A final pH of 9.5 was obtained.

Each mixture obtained was applied to locks of grey hair containing 90% white hairs. After an action time of 30 minutes, the locks were rinsed, washed with a standard shampoo, rinsed again and then dried.

The shades obtained are given in the table below:

| | Example | | | | | | |
|---|---|---|---|---|---|---|---|
| | 77 | 78 | 79 | 80 | 81 | 82 | 83 |
| Shade | yel- | strong | strong | strong | strong | strong | strong |
| ob- | low | violet | red | red- | chro- | blue | violet |
| served | | | | brown | matic | | |
| | | | | | red | | |

### Examples 84 to 96

### Dye Composition Using N-1-(2-diisopropylaminoethyl)-2-methylbenzene-1,4-diamine dihydrochloride (39)

### Examples 84 to 90

### Dyeing in Acidic Medium

The following dye compositions were prepared:

| Example | | | | | | |
|---|---|---|---|---|---|---|
| 84 | 85 | 86 | 87 | 88 | 89 | 90 |

| N-1-(2- | 10⁻³ | mol | 10⁻³ | mol | 10⁻³ | mol | 10⁻³ | mol | 10⁻³ | mol | 10⁻³ | mol | 10⁻³ | mol |
|---|---|---|---|---|---|---|---|---|---|---|---|---|---|---|
| Diisopropylaminoethyl)-2- | | | | | | | | | | | | | | |
| methylbenzene-1,4- | | | | | | | | | | | | | | |
| diamine dihydrochloride | | | | | | | | | | | | | | |
| (39) | | | | | | | | | | | | | | |
| Benzene-1,3-diol | 10⁻³ | mol | | | | | | | | | | | | |
| 5-Amino-2-methylphenol | | | 10⁻³ | mol | | | | | | | | | | |
| 1H-Indol-6-ol | | | | | 10⁻³ | mol | | | | | | | | |
| 2-Aminopyridin-3-ol | | | | | | | 10⁻³ | mol | | | | | | |
| 3,6-Dimethyl-1H- | | | | | | | | | 10⁻³ | mol | | | | |
| pyrazolo[5,1-c]- | | | | | | | | | | | | | | |
| [1,2,4]triazole | | | | | | | | | | | | | | |
| 2-(2,4- | | | | | | | | | | | 10⁻³ | mol | | |
| Diaminophenoxy)ethanol | | | | | | | | | | | | | | |
| hydrochloride | | | | | | | | | | | | | | |
| 3-Amino-2-chloro-6- | | | | | | | | | | | | | 10⁻³ | mol |
| methylphenol | | | | | | | | | | | | | | |
| hydrochloride | | | | | | | | | | | | | | |
| Dye support (1) | (*) | | (*) | | (*) | | (*) | | (*) | | (*) | | (*) | |
| Demineralized water qs | 100 | g | 100 | g | 100 | g | 100 | g | 100 | g | 100 | g | 100 | g |

(*): dye support (1) pH 7

| | | |
|---|---|---|
| 96° ethyl alcohol | 20.8 | g |
| Sodium metabisulfite as an aqueous 35% solution | 0.23 | g A.M. |
| Pentasodium salt of diethylenetriaminepentaacetic acid | 0.48 | g A.M. |
| as an aqueous 40% solution | | |
| C₈-C₁₀ Alkyl polyglucoside as an aqueous 60% solution | 3.6 | g A.M. |
| Benzyl alcohol | 2.0 | g |
| Polyethylene glycol containing 8 ethylene oxide units | 3.0 | g |
| Na₂HPO₄ | 0.28 | g |
| KH₂PO₄ | 0.46 | g |

At the time of use, each composition was mixed with an equal weight of 20-volume aqueous hydrogen peroxide solution (6% by weight). A final pH of 7 was obtained.

Each mixture obtained was applied to locks of grey hair containing 90% white hairs. After an action time of 30 minutes, the locks were rinsed, washed with a standard shampoo, rinsed again and then dried.

The shades obtained are given in the table below:

| | Example | | | | | | |
|---|---|---|---|---|---|---|---|
| | 84 | 85 | 86 | 87 | 88 | 89 | 90 |
| Shade | or- | strong | strong | strong | orange | strong | strong |
| ob- | ange | red- | brown | red | | blue- | red- |
| served | | violet | | | | grey | violet |
| | | | | | | | grey |

### Examples 91 to 96

### Dyeing in Basic Medium

The following dye compositions were prepared:

| Example | | | | | |
|---|---|---|---|---|---|
| 91 | 92 | 93 | 94 | 95 | 96 |

| N-1-(2- | 10⁻³ | mol | 10⁻³ | mol | 10⁻³ | mol | 10⁻³ | mol | 10⁻³ | mol | 10⁻³ | mol |
|---|---|---|---|---|---|---|---|---|---|---|---|---|
| Diisopropylaminoethyl)- | | | | | | | | | | | | |
| 2-methylbenzene-1,4- | | | | | | | | | | | | |
| diamine | | | | | | | | | | | | |
| dihydrochloride (39) | | | | | | | | | | | | |
| 5-Amino-2- | 10⁻³ | mol | | | | | | | | | | |
| methylphenol | | | | | | | | | | | | |
| 1H-Indol-6-ol | | | 10⁻³ | mol | | | | | | | | |
| 2-Aminopyridin-3-ol | | | | | 10⁻³ | mol | | | | | | |
| 3,6-Dimethyl-1H- | | | | | | | 10⁻³ | mol | | | | |
| pyrazolo[5,1- | | | | | | | | | | | | |
| c][1,2,4]triazole | | | | | | | | | | | | |
| 2-(2,4- | | | | | | | | | 10⁻³ | mol | | |
| Diaminophenoxy)- | | | | | | | | | | | | |
| ethanol hydrochloride | | | | | | | | | | | | |
| 3-Amino-2-chloro-6- | | | | | | | | | | | 10⁻³ | mol |
| methylphenol | | | | | | | | | | | | |
| hydrochloride | | | | | | | | | | | | |
| Dye support (2) | (*) | | (*) | | (*) | | (*) | | (*) | | (*) | |
| Demineralized water | 100 | g | 100 | g | 100 | g | 100 | g | 100 | g | 100 | g |
| qs | | | | | | | | | | | | |

(*): dye support (2) pH 9.5

| | | |
|---|---|---|
| 96° ethyl alcohol | 20.8 | g |
| Sodium metabisulfite as an aqueous 35% solution | 0.23 | g A.M. |
| Pentasodium salt of diethylenetriaminepentaacetic acid | 0.48 | g A.M. |
| as an aqueous 40% solution | | |
| C₈-C₁₀ Alkyl polyglucoside as an aqueous 60% solution | 3.6 | g A.M. |
| Benzyl alcohol | 2.0 | g |
| Polyethylene glycol containing 8 ethylene oxide units | 3.0 | g |
| NH₄Cl | 4.32 | g |
| Aqueous ammonia containing 20% NH₃ | 2.94 | g |

| | | |
|---|---|---|
| 96° ethyl alcohol | 20.8 | g |
| Sodium metabisulfite as an aqueous 35% solution | 0.23 | g A.M. |
| Pentasodium salt of diethylenetriaminepentaacetic acid | 0.48 | g A.M. |
| as an aqueous 40% solution | | |
| C₈-C₁₀ Alkyl polyglucoside as an aqueous 60% solution | 3.6 | g A.M. |
| Benzyl alcohol | 2.0 | g |
| Polyethylene glycol containing 8 ethylene oxide units | 3.0 | g |
| NH₄Cl | 4.32 | g |
| Aqueous ammonia containing 20% NH₃ | 2.94 | g |

At the time of use, each composition was mixed with an equal weight of 20-volume aqueous hydrogen peroxide solution (6% by weight). A final pH of 9.5 was obtained.

Each mixture obtained was applied to locks of grey hair containing 90% white hairs. After an action time of 30 minutes, the locks were rinsed, washed with a standard shampoo, rinsed again and then dried.

The shades obtained are given in the table below:

| | Example | | | | | |
|---|---|---|---|---|---|---|
| | 91 | 92 | 93 | 94 | 95 | 96 |
| Shade | red | orange | orange | chromatic | strong blue | strong |
| observed | | | | red | | violet |

### Examples 97 to 102

### Dye Composition Using 2-methyl-N-4-(2-pyrrolidin-1-ylethyl)benzene-1,4-diamine dihydrochloride (31)

### Examples 97 to 99

### Dyeing in Acidic Medium

The following dye compositions were prepared:

| | Example | | | | | |
|---|---|---|---|---|---|---|
| | 97 | | 98 | | 99 | |
| 2-Methyl-N-4-(2-pyrrolidin- | 10⁻³ | mol | 10⁻³ | mol | 10⁻³ | mol |
| 1-ylethyl)benzene-1,4- | | | | | | |
| diamine dihydrochloride | | | | | | |
| (31) | | | | | | |
| 1H-Indol-6-ol | 10⁻³ | mol | | | | |
| 2-(2,4-Diaminophenoxy)- | | | 10⁻³ | mol | | |
| ethanol hydrochloride | | | | | | |
| 3-Amino-2-chloro-6- | | | | | 10⁻³ | mol |
| methylphenol | | | | | | |
| hydrochloride | | | | | | |
| Dye support (1) | (*) | | (*) | | (*) | |
| Demineralized water qs | 100 | g | 100 | g | 100 | g |

(*): dye support (1) pH 7

| | | |
|---|---|---|
| 96° ethyl alcohol | 20.8 | g |
| Sodium metabisulfite as an aqueous 35% solution | 0.23 | g A.M. |
| Pentasodium salt of diethylenetriaminepentaacetic acid | 0.48 | g A.M. |
| as an aqueous 40% solution | | |
| C₈-C₁₀ Alkyl polyglucoside as an aqueous 60% solution | 3.6 | g A.M. |
| Benzyl alcohol | 2.0 | g |
| Polyethylene glycol containing 8 ethylene oxide units | 3.0 | g |
| Na₂HPO₄ | 0.28 | g |
| KH₂PO₄ | 0.46 | g |

| | | |
|---|---|---|
| 96° ethyl alcohol | 20.8 | g |
| Sodium metabisulfite as an aqueous 35% solution | 0.23 | g A.M. |
| Pentasodium salt of diethylenetriaminepentaacetic acid | 0.48 | g A.M. |
| as an aqueous 40% solution | | |
| C₈-C₁₀ Alkyl polyglucoside as an aqueous 60% solution | 3.6 | g A.M. |
| Benzyl alcohol | 2.0 | g |
| Polyethylene glycol containing 8 ethylene oxide units | 3.0 | g |
| Na₂HPO₄ | 0.28 | g |
| KH₂PO₄ | 0.46 | g |

At the time of use, each composition was mixed with an equal weight of 20-volume aqueous hydrogen peroxide solution (6% by weight). A final pH of 7 was obtained.

Each mixture obtained was applied to locks of grey hair containing 90% white hairs. After an action time of 30 minutes, the locks were rinsed, washed with a standard shampoo, rinsed again and then dried.

The shades obtained are given in the table below:

Example

| | 97 | 98 | 99 |
|---|---|---|---|
| Shade observed | orange | strong | red-violet |
| | | blue-grey | grey |

### Examples 100 to 102

### Dyeing in Basic Medium

The following dye compositions were prepared:

| | Example | | | | | |
|---|---|---|---|---|---|---|
| | 100 | | 101 | | 102 | |
| 2-Methyl-N-4-(2-pyrrolidin- | 10⁻³ | mol | 10⁻³ | mol | 10⁻³ | mol |
| 1-ylethyl)benzene-1,4- | | | | | | |
| diamine dihydrochloride | | | | | | |
| (31) | | | | | | |
| 5-Amino-2-methylphenol | 10⁻³ | mol | | | | |
| 2-(2,4-Diaminophenoxy)- | | | 10⁻³ | mol | | |
| ethanol hydrochloride | | | | | | |
| 3-Amino-2-chloro-6- | | | | | 10⁻³ | mol |
| methylphenol | | | | | | |
| hydrochloride | | | | | | |
| Dye support (2) | (*) | | (*) | | (*) | |
| Demineralized water qs | 100 | g | 100 | g | 100 | g |

(*): dye support (2) pH 9.5

| | | |
|---|---|---|
| 96° ethyl alcohol | 20.8 | g |
| Sodium metabisulfite as an aqueous 35% solution | 0.23 | g A.M. |
| Pentasodium salt of diethylenetriaminepentaacetic acid | 0.48 | g A.M. |
| as an aqueous 40% solution | | |
| C₈-C₁₀ Alkyl polyglucoside as an aqueous 60% solution | 3.6 | g A.M. |
| Benzyl alcohol | 2.0 | g |
| Polyethylene glycol containing 8 ethylene oxide units | 3.0 | g |
| NH₄Cl | 4.32 | g |
| Aqueous ammonia containing 20% NH₃ | 2.94 | g |

| | | |
|---|---|---|
| 96° ethyl alcohol | 20.8 | g |
| Sodium metabisulfite as an aqueous 35% solution | 0.23 | g A.M. |
| Pentasodium salt of diethylenetriaminepentaacetic acid | 0.48 | g A.M. |
| as an aqueous 40% solution | | |
| C₈-C₁₀ Alkyl polyglucoside as an aqueous 60% solution | 3.6 | g A.M. |
| Benzyl alcohol | 2.0 | g |
| Polyethylene glycol containing 8 ethylene oxide units | 3.0 | g |
| NH₄Cl | 4.32 | g |
| Aqueous ammonia containing 20% NH₃ | 2.94 | g |

At the time of use, each composition was mixed with an equal weight of 20-volume aqueous hydrogen peroxide solution (6% by weight). A final pH of 9.5 was obtained.

Each mixture obtained was applied to locks of grey hair containing 90% white hairs. After an action time of 30 minutes, the locks were rinsed, washed with a standard shampoo, rinsed again and then dried.

The shades obtained are given in the table below:

Example

| | 100 | 101 | 102 |
|---|---|---|---|
| Shade observed | red | blue-grey | strong |
| | | | violet |

### Examples 103 to 112

### Dye Composition Using 1-[3-(4-amino-2-methylphenylamino)propyl]-pyrrolidin-2-one dihydrochloride (45)

### Examples 103 to 108

### Dyeing in Acidic Medium

The following dye compositions were prepared:

| | Example | | | | | | | | | | | |
|---|---|---|---|---|---|---|---|---|---|---|---|---|
| | 103 | | 104 | | 105 | | 106 | | 107 | | 108 | |
| 1-[3-(4-Amino-2-methyl- | 10⁻³ | mol | 10⁻³ | mol | 10⁻³ | mol | 10⁻³ | mol | 10⁻³ | mol | 10⁻³ | mol |
| phenylamino)propyl]- | | | | | | | | | | | | |
| pyrrolidin-2-one | | | | | | | | | | | | |
| dihydrochloride (45) | | | | | | | | | | | | |
| Benzene-1,3-diol | 10⁻³ | mol | | | | | | | | | | |
| 5-Amino-2-methylphenol | | | 10⁻³ | mol | | | | | | | | |
| 1H-Indol-6-ol | | | | | 10⁻³ | mol | | | | | | |
| 2-Aminopyridin-3-ol | | | | | | | 10⁻³ | mol | | | | |
| 2-(2,4- | | | | | | | | | 10⁻³ | mol | | |
| Diaminophenoxy)ethanol | | | | | | | | | | | | |
| hydrochloride | | | | | | | | | | | | |
| 3-Amino-2-chloro-6- | | | | | | | | | | | 10⁻³ | mol |
| methylphenol | | | | | | | | | | | | |
| hydrochloride | | | | | | | | | | | | |
| Dye support(1) | (*) | | (*) | | (*) | | (*) | | (*) | | (*) | |
| Demineralized water qs | 100 | g | 100 | g | 100 | g | 100 | g | 100 | g | 100 | g |

(*): dye support (1) pH 7

| | | |
|---|---|---|
| 96° ethyl alcohol | 20.8 | g |
| Sodium metabisulfite as an aqueous 35% solution | 0.23 | g A.M. |
| Pentasodium salt of diethylenetriaminepentaacetic acid | 0.48 | g A.M. |
| as an aqueous 40% solution | | |
| C₈-C₁₀ Alkyl polyglucoside as an aqueous 60% solution | 3.6 | g A.M. |
| Benzyl alcohol | 2.0 | g |
| Polyethylene glycol containing 8 ethylene oxide units | 3.0 | g |
| Na₂HPO₄ | 0.28 | g |
| KH₂PO₄ | 0.46 | g |

| | | |
|---|---|---|
| 96° ethyl alcohol | 20.8 | g |
| Sodium metabisulfite as an aqueous 35% solution | 0.23 | g A.M. |
| Pentasodium salt of diethylenetriaminepentaacetic acid | 0.48 | g A.M. |
| as an aqueous 40% solution | | |
| C₈-C₁₀ Alkyl polyglucoside as an aqueous 60% solution | 3.6 | g A.M. |
| Benzyl alcohol | 2.0 | g |
| Polyethylene glycol containing 8 ethylene oxide units | 3.0 | g |
| Na₂HPO₄ | 0.28 | g |
| KH₂PO₄ | 0.46 | g |

At the time of use, each composition was mixed with an equal weight of 20-volume aqueous hydrogen peroxide solution (6% by weight). A final pH of 7 was obtained.

Each mixture obtained was applied to locks of grey hair containing 90% white hairs. After an action time of 30 minutes, the locks were rinsed, washed with a standard shampoo, rinsed again and then dried.

The shades obtained were given in the table below:

| | Example | | | | | |
|---|---|---|---|---|---|---|
| | 103 | 104 | 105 | 106 | 107 | 108 |
| Shade | yellow- | strong | strong | brown | strong | strong |
| observed | brown | violet | brown | | blue- | blue- |
| | | grey | | | green | violet |

### Examples 109 to 112

### Dyeing in Basic Medium

The following dye compositions were prepared:

| | Example | | | | | | | |
|---|---|---|---|---|---|---|---|---|
| | 109 | | 110 | | 111 | | 112 | |
| 1-[3-(4-Amino-2-methylphenylamino) | 10⁻³ | mol | 10⁻³ | mol | 10⁻³ | mol | 10⁻³ | mol |
| propyl]-pyrrolidin-2-one | | | | | | | | |
| dihydrochloride (45) | | | | | | | | |
| 5-Amino-2-methylphenol | 10⁻³ | mol | | | | | | |
| 3,6-Dimethyl-1H- | | | 10⁻³ | mol | | | | |
| pyrazolo[5,1-c]-[1,2,4]triazole | | | | | | | | |
| 2-(2,4-Diaminophenoxy)- | | | | | 10⁻³ | mol | | |
| ethanol hydrochloride | | | | | | | | |
| 3-Amino-2-chloro-6- | | | | | | | 10⁻³ | mol |
| methylphenol hydrochloride | | | | | | | | |
| Dye support (2) | (*) | | (*) | | (*) | | (*) | |
| Demineralized water qs | 100 | g | 100 | g | 100 | g | 100 | g |

(*): dye support (2) pH 9.5

| | | |
|---|---|---|
| 96° ethyl alcohol | 20.8 | g |
| Sodium metabisulfite as an aqueous 35% solution | 0.23 | g A.M. |
| Pentasodium salt of diethylenetriaminepentaacetic acid | 0.48 | g A.M. |
| as an aqueous 40% solution | | |
| C₈-C₁₀ Alkyl polyglucoside as an aqueous 60% solution | 3.6 | g A.M. |
| Benzyl alcohol | 2.0 | g |
| Polyethylene glycol containing 8 ethylene oxide units | 3.0 | g |
| NH₄Cl | 4.32 | g |
| Aqueous ammonia containing 20% NH₃ | 2.94 | g |

| | | |
|---|---|---|
| 96° ethyl alcohol | 20.8 | g |
| Sodium metabisulfite as an aqueous 35% solution | 0.23 | g A.M. |
| Pentasodium salt of diethylenetriaminepentaacetic acid | 0.48 | g A.M. |
| as an aqueous 40% solution | | |
| C₈-C₁₀ Alkyl polyglucoside as an aqueous 60% solution | 3.6 | g A.M. |
| Benzyl alcohol | 2.0 | g |
| Polyethylene glycol containing 8 ethylene oxide units | 3.0 | g |
| NH₄Cl | 4.32 | g |
| Aqueous ammonia containing 20% NH₃ | 2.94 | g |

At the time of use, each composition was mixed with an equal weight of 20-volume aqueous hydrogen peroxide solution (6% by weight). A final pH of 9.5 was obtained.

Each mixture obtained was applied to locks of grey hair containing 90% white hairs. After an action time of 30 minutes, the locks were rinsed, washed with a standard shampoo, rinsed again and then dried.

The shades obtained are given in the table below:

Example

| | 109 | 110 | 111 | 112 |
|---|---|---|---|---|
| Shade | violet | chromatic | strong | strong |
| observed | | red | blue | blue-violet |

### Examples 112 to 118

### Dye Composition Using 3-methyl-N-4-(2-pyrrolidin-1-ylethyl)benzene-1,4-diamine dihydrochloride (47)

### Examples 112 to 114

### Dyeing in Acidic Medium

The following dye compositions were prepared:

| | Example | | | | | |
|---|---|---|---|---|---|---|
| | 112 | | 113 | | 114 | |
| 3-Methyl-N-4-(2-pyrrolidin- | 10⁻³ | mol | 10⁻³ | mol | 10⁻³ | mol |
| 1-ylethyl)benzene-1,4- | | | | | | |
| diamine dihydrochloride | | | | | | |
| (47) | | | | | | |
| 1H-Indol-6-ol | 10⁻³ | mol | | | | |
| 2-(2,4-Diaminophenoxy)- | | | 10⁻³ | mol | | |
| ethanol hydrochloride | | | | | | |
| 3-Amino-2-chloro-6- | | | | | 10⁻³ | mol |
| methylphenol | | | | | | |
| hydrochloride | | | | | | |
| Dye support(1) | (*) | | (*) | | (*) | |
| Demineralized water qs | 100 | g | 100 | g | 100 | g |

(*): dye support (1) pH 7

| | |
|---|---|
| 96° ethyl alcohol | 20.8 g |
| Sodium metabisulfite as an aqueous 35% solution | 0.23 g A.M. |
| Pentasodium salt of diethylenetriaminepentaacetic acid | 0.48 g A.M. |
| as an aqueous 40% solution | |
| C₈-C₁₀ Alkyl polyglucoside as an aqueous 60% solution | 3.6 g A.M. |
| Benzyl alcohol | 2.0 g |
| Polyethylene glycol containing 8 ethylene oxide units | 3.0 g |
| Na₂HPO₄ | 0.28 g |
| KH₂PO₄ | 0.46 g |

At the time of use, each composition was mixed with an equal weight of 20-volume aqueous hydrogen peroxide solution (6% by weight). A final pH of 7 was obtained.

Each mixture obtained was applied to locks of grey hair containing 90% white hairs. After an action time of 30 minutes, the locks were rinsed, washed with a standard shampoo, rinsed again and then dried.

The shades obtained are given in the table below:

| | Example | | |
|---|---|---|---|
| | 112 | 113 | 114 |
| Shade observed | red-brown | strong blue- | red-violet |
| | | grey | grey |

### Examples 115 to 118

### Dyeing in Basic Medium

The following dye compositions were prepared:

| | Example | | | | | | | |
|---|---|---|---|---|---|---|---|---|
| | 115 | | 116 | | 117 | | 118 | |
| 3-Methyl-N-4-(2-pyrrolidin-1- | 10⁻³ | mol | 10⁻³ | mol | 10⁻³ | mol | 10⁻³ | mol |
| ylethyl)benzene-1,4-diamine | | | | | | | | |
| dihydrochloride (47) | | | | | | | | |
| 5-Amino-2-methylphenol | 10⁻³ | mol | | | | | | |
| 3,6-Dimethyl-1H-pyrazolo[5,1- | | | 10⁻³ | mol | | | | |
| c]-[1,2,4]triazole | | | | | | | | |
| 2-(2,4-Diaminophenoxy)ethanol | | | | | 10⁻³ | mol | | |
| hydrochloride | | | | | | | | |
| 3-Amino-2-chloro-6- | | | | | | | 10⁻³ | mol |
| methylphenol hydrochloride | | | | | | | | |
| Dye support(2) | (*) | | (*) | | (*) | | (*) | |
| Demineralized water qs | 100 | g | 100 | g | 100 | g | 100 | g |

(*): dye support (2) pH 9.5

| | |
|---|---|
| 96° ethyl alcohol | 20.8 g |
| Sodium metabisulfite as an aqueous 35% solution | 0.23 g A.M. |
| Pentasodium salt of diethylenetriaminepentaacetic acid | 0.48 g A.M. |
| as an aqueous 40% solution | |
| C₈-C₁₀ Alkyl polyglucoside as an aqueous 60% solution | 3.6 g A.M. |
| Benzyl alcohol | 2.0 g |
| Polyethylene glycol containing 8 ethylene oxide units | 3.0 g |
| NH₄Cl | 4.32 g |
| Aqueous ammonia containing 20% NH₃ | 2.94 g |

At the time of use, each composition was mixed with an equal weight of 20-volume aqueous hydrogen peroxide solution (6% by weight). A final pH of 9.5 was obtained.

Each mixture obtained was applied to locks of grey hair containing 90% white hairs. After an action time of 30 minutes, the locks were rinsed, washed with a standard shampoo, rinsed again and then dried.

The shades obtained are given in the table below:

Example

| | 115 | 116 | 117 | 118 |
|---|---|---|---|---|
| Shade observed | red | chromatic | strong | strong |
| | | red | blue | violet |

### Examples 119 to 126

### Dye Composition Using 3-methyl-N-1-(3-pyrrolidin-1-ylpropyl)benzene-1,4-diamine dihydrochloride (43)

### Examples 119 to 123

### Dyeing in Acidic Medium

The following dye compositions were prepared:

| | Example | | | | | | | | | |
|---|---|---|---|---|---|---|---|---|---|---|
| | 119 | | 120 | | 121 | | 122 | | 123 | |
| 3-Methyl-N-1-(3-pyrrolidin- | 10⁻³ | mol | 10⁻³ | mol | 10⁻³ | mol | 10⁻³ | mol | 10⁻³ | mol |
| 1-ylpropyl)benzene-1,4- | | | | | | | | | | |
| diamine dihydrochloride | | | | | | | | | | |
| (43) | | | | | | | | | | |
| 5-Amino-2-methylphenol | 10⁻³ | mol | | | | | | | | |
| 1H-Indol-6-ol | | | 10⁻³ | mol | | | | | | |
| 2-Aminopyridin-3-ol | | | | | 10⁻³ | mol | | | | |
| 2-(2,4-Diaminophenoxy)- | | | | | | | 10⁻³ | mol | | |
| ethanol hydrochloride | | | | | | | | | | |
| 3-Amino-2-chloro-6- | | | | | | | | | 10⁻³ | mol |
| methylphenol | | | | | | | | | | |
| hydrochloride | | | | | | | | | | |
| Dye support(1) | (*) | | (*) | | (*) | | (*) | | (*) | |
| Demineralized water qs | 100 | g | 100 | g | 100 | g | 100 | g | 100 | g |

(*): dye support (1) pH 7

| | | |
|---|---|---|
| 96° ethyl alcohol | 20.8 | g |
| Sodium metabisulfite as an aqueous 35% solution | 0.23 | g A.M. |
| Pentasodium salt of diethylenetriaminepentaacetic acid | 0.48 | g A.M. |
| as an aqueous 40% solution | | |
| C₈-C₁₀ Alkyl polyglucoside as an aqueous 60% | 3.6 | g A.M. |
| solution | | |
| Benzyl alcohol | 2.0 | g |
| Polyethylene glycol containing 8 ethylene oxide units | 3.0 | g |
| Na₂HPO₄ | 0.28 | g |
| KH₂PO₄ | 0.46 | g |

At the time of use, each composition was mixed with an equal weight of 20-volume aqueous hydrogen peroxide solution (6% by weight). A final pH of 7 was obtained.

Each mixture obtained was applied to locks of grey hair containing 90% white hairs. After an action time of 30 minutes, the locks were rinsed, washed with a standard shampoo, rinsed again and then dried.

The shades obtained are given in the table below:

| | Example | | | | |
|---|---|---|---|---|---|
| | 119 | 120 | 121 | 122 | 123 |
| Shade | red-violet | orange | orange- | strong blue- | strong |
| observed | grey | | brown | green grey | blue-violet |

### Examples 123 to 126

### Dyeing in Basic Medium

The following dye compositions were prepared:

| | Example | | | | | | | |
|---|---|---|---|---|---|---|---|---|
| | 123 | | 124 | | 125 | | 126 | |
| 3-Methyl-N-1-(3-pyrrolidin-1- | 10⁻³ | mol | 10⁻³ | mol | 10⁻³ | mol | 10⁻³ | mol |
| ylpropyl)benzene-1,4-diamine | | | | | | | | |
| dihydrochloride (43) | | | | | | | | |
| 5-Amino-2-methylphenol | 10⁻³ | mol | | | | | | |
| 3,6-Dimethyl-1H-pyrazolo[5,1- | | | 10⁻³ | mol | | | | |
| c]-[1,2,4]triazole | | | | | | | | |
| 2-(2,4-Diaminophenoxy)ethanol | | | | | 10⁻³ | mol | | |
| hydrochloride | | | | | | | | |
| 3-Amino-2-chloro-6- | | | | | | | 10⁻³ | mol |
| methylphenol hydrochloride | | | | | | | | |
| Dye support(2) | (*) | | (*) | | (*) | | (*) | |
| Demineralized water qs | 100 | g | 100 | g | 100 | g | 100 | g |

(*): dye support (2) pH 9.5

| | |
|---|---|
| 96° ethyl alcohol | 20.8 g |
| Sodium metabisulfite as an aqueous 35% solution | 0.23 g A.M. |
| Pentasodium salt of diethylenetriaminepentaacetic acid | 0.48 g A.M. |
| as an aqueous 40% solution | |
| C₈-C₁₀ Alkyl polyglucoside as an aqueous 60% | 3.6 g A.M. |
| solution | |
| Benzyl alcohol | 2.0 g |
| Polyethylene glycol containing 8 ethylene oxide units | 3.0 g |
| NH₄Cl | 4.32 g |
| Aqueous ammonia containing 20% NH₃ | 2.94 g |

At the time of use, each composition was mixed with an equal weight of 20-volume aqueous hydrogen peroxide solution (6% by weight). A final pH of 9.5 was obtained.

Each mixture obtained was applied to locks of grey hair containing 90% white hairs. After an action time of 30 minutes, the locks were rinsed, washed with a standard shampoo, rinsed again and then dried.

The shades obtained are given in the table below:

Example

| | 123 | 124 | 125 | 126 |
|---|---|---|---|---|
| Shade observed | violet | chromatic | blue- | strong |
| | | red | green | blue- |
| | | | | violet |

What is claimed is:

1. An ortho-substituted and/or meta-substituted N-alkylamino secondary para-phenylenediamine compound of formula (I): 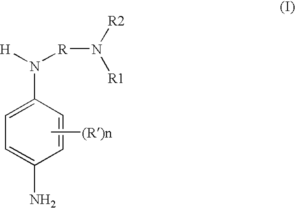
wherein:
R₁ and R₂, which may be identical or different, are each chosen from a hydrogen atom and alkyl groups, or the radicals R₁ and R₂ form, together with the nitrogen on which they are attached, a saturated 5- or 6-membered ring optionally interrupted with at least one entity chosen from nitrogen atoms and carbonyl groups, and optionally substituted with at least one alkyl group;
R is chosen from C₂-C₁₀ alkylene radicals, optionally interrupted with at least one atom chosen from nitrogen and oxygen, and optionally substituted with at least one group chosen from amino, monoalkylamino, dialkylamino, alkylcarbonyl, amido, alkoxycarbonyl, monoalkylaminocarbonyl, and dialkylaminocarbonyl groups;
R′ is chosen from alkyl, alkoxy, alkoxyalkyl, monohydroxyalkyl and polyhydroxyalkyl radicals, and a chlorine atom; and
n is an integer equal to 1;
with the proviso that the compound of formula (I) is not chosen from N-1-(2-diethylaminoethyl)-2-methoxy-benzene-1,4-diamine; N-1-(3-diethylaminopropyl)-3-methylbenzene-1,4-diamine, and N-1-(3-pyrrolidino-propyl)-3-methyl-benzene-1,4 diamine.

2. The ortho-substituted and/or meta-substituted N-alkylamino secondary para-phenylenediamine compound of formula (I) according to claim 1, wherein R₁ is chosen from a hydrogen atom and alkyl groups and R₂ is chosen from a hydrogen atom and alkyl groups.

3. The ortho-substituted and/or meta-substituted N-alkylamino secondary para-phenylenediamine compounds of formula (I) according to claim 1, wherein R₁ is chosen from a hydrogen atom and R₂ is chosen from a hydrogen atom and alkyl groups.

4. The ortho-substituted and/or meta-substituted N-alkylamino secondary para-phenylenediamine compound of formula (I) according to claim 1, wherein R₁ and R₂ both chosen from alkyl groups.

5. The ortho-substituted and/or meta-substituted N-alkylamino secondary para-phenylenediamine compound of formula (I) according to claim 4, wherein R₁ and R₂ are both chosen from two identical alkyl groups.

6. The ortho-substituted and/or meta-substituted N-alkylamino secondary para-phenylenediamine compound of formula (I) according to claim 1, wherein R₁ and R₂ form, together with the nitrogen to which they are attached, a saturated 5- or 6-membered ring optionally interrupted with at least one atom chosen from nitrogen and oxygen and optionally substituted with at least one alkyl group.

7. The ortho-substituted and/or meta-substituted N-alkylamino secondary para-phenylenediamine compound of formula (I) according to claim 6, wherein R₁ and R₂ form, together with the nitrogen atom to which they are attached, a saturated 5- or 6-membered ring optionally interrupted with at least one nitrogen atom, or optionally substituted with at least one C₁-C₃ alkyl group.

8. The ortho-substituted and/or meta-substituted N-alkylamino secondary para-phenylenediamine compound of formula (I) according to claim 7, wherein R is chosen from linear and branched C₂-C₈ alkylene radicals optionally interrupted with one or two nitrogen atoms.

9. The ortho-substituted and/or meta-substituted N-alkylamino secondary para-phenylenediamine compound of formula (I) according to claim 1, wherein R′ is chosen from C₁-C₃ alkyl, C₁-C₃ alkoxy and (C₁-C₃)alkoxy(C₁-C₃)alkyl groups and a chlorine atom.

10. The ortho-substituted and/or meta-substituted N-alkylamino secondary para-phenylenediamine compound of formula (I) according to claim 1, wherein the compound of formula (I) is chosen from: N-1-(2-aminoethyl)-2-methoxymethylbenzene-1,4-diamine, 1-[3-(4-amino-2-methylphenylamino)propyl]pyrrolidin-2-one; 2-methyl-N-1-[3-(2-methylpiperid-1-yl)propyl]benzene-1,4-diamine; N-1-(4-diethylamino-1-methylbutyl)-2-methylbenzene-1,4-diamine; N-1-(3-dibutylaminopropyl)-2-methylbenzene-1,4-diamine; N-1-(2-dimethylamino-1-methylethyl)-2-methylbenzene-1,4-diamine; N-1-(2-diisopropylaminoethyl)-2-methylbenzene-1,4-diamine; N-1-(2-dibutylaminoethyl)-2-methylbenzene-1,4-diamine; 2-methyl-N-1-(3-pyrrolidin-1-ylpropyl)benzene-1,4-diamine; 2-methyl-N-1-(2-pyrrolidin-1-ylethyl)benzene-1,4-diamine; N-1-(4-aminobutyl)-2-methylbenzene-1,4-diamine; N-1-(2-isopropylaminoethyl)-2-methylbenzene-1,4-diamine; N-1-(2-dimethylaminoethyl)-2-methylbenzene-1,4-diamine; N-1-(3-dimethylaminopropyl)-2-methylbenzene-1,4-diamine; N-1-(8-aminooctyl)-2-methylbenzene-1,4-diamine; N-1-(6-aminohexyl)-2-methylbenzene-1,4-diamine; N-1-{2-[2-(2-dimethylaminoethoxy)ethoxy]ethyl}-2-methylbenzene-1,4-diamine; 2-methyl-N-1-[3-(4-methylpiperazin-1-yl)propyl]benzene-1,4-diamine; 1-[3-(4-amino-3-methyl-phenylamino)-propyl]-pyrrolidin-2-one; N-1-(3-diethylamino-propyl)-2-methylbenzene-1,4-diamine; N-1-(3-dimethylamino-2,2-dimethyl-propyl)-2-methylbenzene-1,4-diamine; 2-methyl-N-1-(2-piperid-1-ylethyl)benzene-1,4-diamine; 3-methyl-N-1-[3-(2-methylpiperid-1-yl)propyl]benzene-1,4-diamine; N-1-(4-diethylamino-1-methylbutyl)-3-methylbenzene-1,4-diamine; N-1-(3-dibutylaminopropyl)-2-methylbenzene-1,4-diamine; N−1-(2-dimethylamino-1-methylethyl)-3-methylbenzene-1,4-diamine; N-1-(2-diisopropylaminoethyl)-3-methylbenzene-1,4-diamine; N-1-(2-dibutylaminoethyl)-3-methylbenzene-1,4-diamine; 3-methyl-N-1-(3-pyrrolidin-1-ylpropyl) benzene-1,4-diamine; 3-methyl-N-1-(2-pyrrolidin-1-ylethyl)benzene-1,4-diamine; N-1-(4-aminobutyl)-3-methylbenzene-1,4-diamine; N-1-(2-isopropylaminoethyl)-3-methylbenzene-1,4-diamine; N-1-(2-dimethylaminoethyl)-3-methylbenzene-1,4-diamine; N-1-(3-dimethylaminopropyl)-3-methylbenzene-1,4-diamine; N-1-(8-aminooctyl)-3-methylbenzene-1,4-diamine; N-1-(6-aminohexyl)-3-methylbenzene-1,4-diamine; N-1-{2-[2-(2-dimethylaminoethoxy)ethoxy]ethyl}-3-methylbenzene-1,4-diamine; 3-methyl-N-1-[3-(4-methylpiperazin-1-yl)propyl]benzene-1,4-diamine; N-1-(3-diethylaminopropyl)-3-methylbenzene-1,4-diamine; N-1-(3-dimethylamino-2,2-dimethylpropyl)-3-methylbenzene-1,4-diamine; 2-methyl-N-4-(2-piperid-1-ylethyl)benzene-1,4-diamine; N-1-(2-aminoethyl)-2-methylbenzene-1,4-diamine; and N-4-(2-aminoethyl)-2-methylbenzene-1,4-diamine.

11. The ortho-substituted and/or meta-substituted N-alkylamino secondary para-phenylenediamine compound of formula (I) according to claim 1, wherein the compound of formula (I) is in the form of a salt.

12. The ortho-substituted and/or meta-substituted N-alkylamino secondary para-phenylenediamine compound of formula (I) according to claim 11, wherein the compound of formula (I) is in the form of an acid addition salt chosen from hydrochlorides, hydrobromides, sulfates, citrates, succinates, tartrates, lactates, tosylates, benzenesulfonates, phosphates, and acetates.

13. A nitro compound of formula (II): 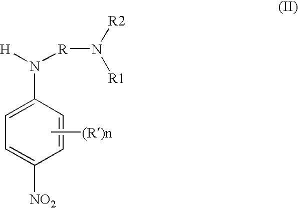
wherein:
R₁ and R₂, which may be identical or different, are each chosen from a hydrogen atom and alkyl groups, and radicals R₁ and R₂ form, together with the nitrogen atom to which they are attached, a saturated 5- or 6-membered ring optionally interrupted with at least one entity chosen from nitrogen and carbonyl groups, and optionally substituted with at least one alkyl group;
R is chosen from linear and branched C₂-C₁₀ alkylene radicals, optionally interrupted with at least one atom chosen from nitrogen and oxygen, and optionally substituted with at least one group chosen from amino, monoalkylamino, dialkylamino, alkylcarbonyl, amido, alkoxycarbonyl, monoalkylaminocarbonyl and dialkylaminocarbonyl groups;
R′ is chosen from alkyl, alkoxy, alkoxyalkyl, monohydroxyalkyl and polyhydroxyalkyl radicals and a chlorine atom; and
n is an integer equal to 1,
with the proviso that the compound of formula II is not N-1-(3-diethylaminopropyl)-3-methyl-4-nitro-1-aminobenzene and N-1 (3-pyrrolidino-propyl)-3-methyl-4-nitro-1-aminobenzene

14. The ortho-substituted and/or meta-substituted N-alkylamino secondary para-phenylenediamine compound of formula (I) according to claim 1, wherein the compound of formula (I) is prepared by performing a reduction reaction with the corresponding nitro compound.

15. A composition for dyeing fibers comprising, in a medium suitable for dyeing, at least one oxidation base chosen from ortho-substituted and meta-substituted N-alkylamino secondary para-phenylenediamine compounds of formula (I) below, 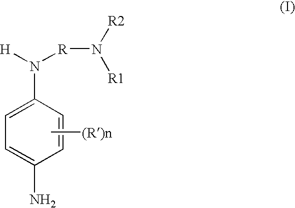
wherein:
R₁ and R₂, which may be identical or different, are each chosen from a hydrogen atom and alkyl groups, or the radicals R₁ and R₂ form, together with the nitrogen on which they are substituent, a saturated 5- or 6-membered ring optionally interrupted with at least one entity chosen from nitrogen atoms and carbonyl groups, and optionally substituted with at least one alkyl group;
R is chosen from C₂-C₁₀ alkylene radicals, optionally interrupted with at least one atom chosen from nitrogen and oxygen, and optionally substituted with at least one group chosen from amino, monoalkylamino, dialkylamino, alkylcarbonyl, amido, alkoxycarbonyl, monoalkylaminocarbonyl, and dialkylaminocarbonyl groups;
R′ is chosen from alkyl, alkoxy, alkoxyalkyl, monohydroxyalkyl and polyhydroxyalkyl radicals, and a chlorine atom; and
n is an integer equal to 1,
with the proviso that the compound of formula (I) is not chosen from N-1-(2-diethylaminoethyl)-2-methoxy-benzene-1,4-diamine; N-1-(3-diethylaminopropyl)-3-methylbenzene-1,4-diamine, and N-1-(3-pyrrolidino-propyl)-3-methyl-benzene-1,4 diamine.

16. The composition according to claim 15, further comprising at least one cosmetic adjuvant chosen from antioxidants, penetrating agents, sequestering agents, fragrances, buffers, dispersants, surfactants, conditioning agents, film-forming agents, polymers, ceramides, preserving agents, nacreous agents, opacifiers, vitamins and provitamins.

17. The composition according to claim 15, wherein the at least one oxidation base chosen from ortho-substituted and meta-substituted N-alkylamino secondary para-phenylenediamine compounds of formula (I) is present in an amount ranging from 0.0001% to 20% by weight, relative to the total weight of the composition.

18. The composition according to claim 17, wherein the at least one oxidation base chosen from ortho-substituted and meta-substituted N-alkylamino secondary para-phenylenediamine compounds of formula (I) is present in an amount ranging from 0.005% to 6% by weight, relative to the total weight of the composition.

19. The composition according claim 15, wherein the medium that is suitable for dyeing is chosen from water and mixtures of water and at least one organic solvent.

20. The composition according to claim 16, wherein the at least one cosmetic adjuvant is present in an amount ranging from 0.01% to 20% by weight, relative to the weight of the composition.

21. The composition according to claim 15, further comprising at least one coupler.

22. The composition according to claim 21, wherein the at least one coupler is chosen from meta-phenylenediamines, meta-aminophenols, meta-diphenols, naphthalene-based couplers, heterocyclic couplers, and addition salts thereof.

23. The composition according to claim 22, wherein the at least one coupler is present in an amount ranging from 0.0001% to 20% by weight, relative to the total weight of the composition.

24. The composition according to claim 15, further comprising at least one additional oxidation base other than the at least one oxidation base chosen from ortho-substituted and meta-substituted N-alkylamino secondary para-phenylenediamine compounds of formula (I), chosen from para-phenylenediamines, bis(phenyl)alkylenediamines, para-aminophenols, ortho-aminophenols, heterocyclic bases, and addition salts thereof.

25. The composition according to claim 24, wherein the at least one additional oxidation base is present in an amount ranging from 0.0001% to 20% by weight, relative to the total weight of the composition.

26. The composition according to claim 15, further comprising at least one direct dye chosen from natural and cationic direct dyes.

27. The composition according to claim 15, wherein the composition is in the form of a ready-to-use composition and further comprises at least one oxidizing agent chosen from hydrogen peroxide, urea peroxide, alkaline metal bromates, persalts, peracids and oxidase enzymes.

28. A process for dyeing keratin fibers comprising,
applying to the fibers, in the presence of at least one oxidizing agent, at least one composition comprising, in a medium suitable for dyeing, at least one oxidation base chosen from ortho-substituted and meta-substituted N-alkylamino secondary para-phenylenediamine compounds of formula (I) below, 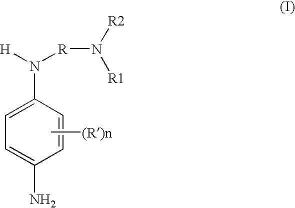
wherein:
R₁ and R₂, which may be identical or different, are each chosen from a hydrogen atom and alkyl groups, or the radicals R₁ and R₂ form, together with the nitrogen on which they are substituent, a saturated 5- or 6-membered ring optionally interrupted with at least one entity chosen from nitrogen atoms and carbonyl groups, and optionally substituted with at least one alkyl group;
R is chosen from C₂-C₁₀ alkylene radicals, optionally interrupted with at least one atom chosen from nitrogen and oxygen, and optionally substituted with at least one group chosen from amino, monoalkylamino, dialkylamino, alkylcarbonyl, amido, alkoxycarbonyl, monoalkylaminocarbonyl, and dialkylaminocarbonyl groups;
R′ is chosen from alkyl, alkoxy, alkoxyalkyl, monohydroxyalkyl and polyhydroxyalkyl radicals, and a chlorine atom; and
n is an integer equal to 1,
with the proviso that the compound of formula (I) is not chosen from N-1-(2-diethylaminoethyl)-2-methoxy-benzene-1,4-diamine; N-1-(3-diethylaminopropyl)-3-methylbenzene-1,4-diamine, and N-1-(3-pyrrolidino-propyl)-3-methyl-benzene-1,4 diamine.
leaving the at least one composition on the fibers for a time that is sufficient to develop the desired coloration; and
applying the at least one oxidizing agent before, simultaneously with or after applying the at least one composition.

29. A process for dyeing keratin fibers comprising,
applying to the fibers at least one ready-to-use composition comprising, in a medium suitable for dyeing,
at least one ortho-substituted and meta-substituted N-alkylamino secondary para-phenylenediamine compound of formula (I) below, 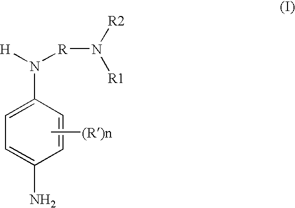
wherein:
R₁ and R₂, which may be identical or different, are each chosen from a hydrogen atom and alkyl groups, or the radicals R₁ and R₂ form, together with the nitrogen on which they are substituent, a saturated 5- or 6-membered ring optionally interrupted with at least one entity chosen from nitrogen atoms and carbonyl groups, and optionally substituted with at least one alkyl group;
R is chosen from C₂-C₁₀ alkylene radicals, optionally interrupted with at least one atom chosen from nitrogen and oxygen, and optionally substituted with at least one group chosen from amino, monoalkylamino, dialkylamino, alkylcarbonyl, amido, alkoxycarbonyl, monoalkylaminocarbonyl, and dialkylaminocarbonyl groups;
R′ is chosen from alkyl, alkoxy, alkoxyalkyl, monohydroxyalkyl and polyhydroxyalkyl radicals, and a chlorine atom; and
n is an integer equal to 1,
with the proviso that the compound of formula (I) is not chosen from N−1-(2-diethylaminoethyl)-2-methoxy-benzene-1,4-diamine; N-1-(3-diethylaminopropyl)-3-methylbenzene-1,4-diamine, and N-1-(3-pyrrolidino-propyl)-3-methyl-benzene-1,4 diamine and
at least one oxidizing agent chosen from hydrogen peroxide, urea peroxide, alkaline metal bromates, persalts, peracids and oxidase enzymes; and
leaving the at least one ready-to-use composition on the fibers for a time that is sufficient to develop the desired coloration.

30. A multicompartment kit or device comprising,
at least one first compartment comprising at least one composition comprising, in a medium suitable for dyeing, at least one oxidation base chosen from ortho-substituted and meta-substituted N-alkylamino secondary para-phenylenediamine compound of formula (I) below, 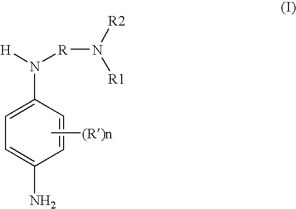
wherein:
R₁ and R₂, which may be identical or different, are each chosen from a hydrogen atom and alkyl groups, or the radicals R₁ and R₂ form, together with the nitrogen on which they are substituent, a saturated 5- or 6-membered ring optionally interrupted with at least one entity chosen from nitrogen atoms and carbonyl groups, and optionally substituted with at least one alkyl group;
R is chosen from C₂-C₁₀ alkylene radicals, optionally interrupted with at least one atom chosen from nitrogen and oxygen, and optionally substituted with at least one group chosen from amino, monoalkylamino, dialkylamino, alkylcarbonyl, amido, alkoxycarbonyl, monoalkylaminocarbonyl, and dialkylaminocarbonyl groups;
R′ is chosen from alkyl, alkoxy, alkoxyalkyl, monohydroxyalkyl and polyhydroxyalkyl radicals, and a chlorine atom; and
n is an integer equal to 1,
with the proviso that the compound of formula (I) is not chosen from N-1-(2-diethylaminoethyl)-2-methoxy-benzene-1,4-diamine; N-1-(3-diethylaminopropyl)-3-methylbenzene-1,4-diamine, and N-1-(3-pyrrolidino-propyl)-3-methyl-benzene-1,4 diamine, and
at least one second compartment comprising at least one oxidizing agent.
